(19) United States
(12) Patent Application Publication
(10) Pub. No.: US 2012/0058932 A1
(43) Pub. Date: Mar. 8, 2012

(54) ACTIVE INGREDIENT-PEPTIDE CONSTRUCT FOR EXTRACELLULAR CONCENTRATION

(75) Inventors: Gunter Fischer, Halle (DE); Miroslav Malesevic, Halle (DE); Frank Erdmann, Halle (DE); Jan Kühling, Halle (DE); Michael Ilya Bukrinsky, Potomac, MD (US); Stephanie Constant, Herndon, VA (US)

(73) Assignee: MAX PLANCK-GESELLSCHAFT ZUR FORDERUNG DER WISSENSCHAFTEN E.V., Muenchen (DE)

(21) Appl. No.: 13/132,695

(22) PCT Filed: Dec. 4, 2009
(86) PCT No.: PCT/EP2009/008683

§ 371 Date: Nov. 21, 2011

(30) Foreign Application Priority Data
Dec. 4, 2008 (DE) 10 2008 060 549.2

(51) Int. Cl. A61K 38/00; C07K 5/00; C07K 7/06; C07D 498/18; C07K 14/00; C07K 5/06; C07K 5/08; C07K 5/10; C07K 2/00; C07K 7/08

(52) U.S. Cl. 514/1.1; 530/300; 530/330; 530/329; 530/328; 530/327; 530/326; 530/325; 530/324; 560/155; 530/317; 540/456

(57) ABSTRACT

The present invention relates to an active ingredient-peptide construct for extracellular concentration, a process for the concentration of active ingredients in an extracellular space of a multicellular object, the use of the active ingredient-peptide construct according to the invention for the production of a medicinal product and a pharmaceutical composition containing the active ingredient-peptide construct according to the invention.

The present invention relates to an active ingredient-peptide construct for extracellular concentration, a process for the concentration of active ingredients in an extracellular space of a multicellular object, the use of the active ingredient-peptide construct according to the invention for the production of a medicinal product and a pharmaceutical composition containing the active ingredient-peptide construct according to the invention.

The effect of biologically effective molecules, so-called “active ingredients”, which are generally pharmaceutical active ingredients, develops mostly both inside as well as outside biological cells. Hitherto, primarily the problem has occurred that active ingredients whose effect is to develop only inside the cell cause undesired changes in the extracellular space even before passing through the cell membrane. The problem that additionally arises here is that one and the same active ingredient can develop a different effect inside and outside the cell. The actual effect thus comprises two components—the desired intracellular and the undesired extracellular one. If the intracellular effect is to be achieved, the extracellular (side-)effect also often had to be accepted, because the transport to the cell generally includes the crossing of an extracellular space.

An equally important problem, but one not hitherto described in the state of the art, is the administration of active ingredients which are supposed to or should develop their effect outside the cell only. Above all in medicine a range of active ingredients is known which not only do not develop the desired effect in the cell but actually have a toxic effect or are harmful in some other way. To this is added the fact that, to achieve a specific extracellular effect, a much higher dose must be administered than is actually required in order to compensate for the “loss” of the active ingredients which have migrated into the inside of the cell.

Active ingredients can act from outside the cell on molecules or structures. Such biological molecules of the extracellular space can for example be: enzymes, inhibitors, activators or receptors. By “structures” is meant for example the extracellular matrix which is formed from the totality of the macromolecules which are found outside the plasma membrane of cells in tissues and organs.

The object of the present invention was therefore to find a way in which active ingredients can be administered to a multicellular object without the administered active ingredients being able to penetrate into the inside of the cell of the multicellular object. Specifically it was an object of the present invention to find a way in which active ingredients can also be made usable for therapies whose field of use—above all in the case of active ingredients which develop a toxic effect intracellularly—is strictly limited, or must remain limited, to the extracellular space,

It was surprisingly found that the entry of active ingredients into cells can be prevented if these are administered in the form of an active ingredient-peptide construct with a net charge that is negative at pH 6 and the active ingredient-peptide construct is free from a constituent that can pass through the membrane of a biological cell.

However, the active ingredient-peptide construct according to the invention not only has the advantage that active ingredients can be used selectively in an extracellular space, but also offers, through the use of specially composed peptides, the possibility of selectively compensating for further disadvantages which are associated with the use of specific active ingredients. For example, the effectiveness of active ingredients which are difficult to dissolve in water and thus in most extracellular tissues can be improved by bonding with peptides which also have a very high water solubility. This has the further associated advantage that in turn the quantity of active ingredient to be used can be reduced.

The object of the present invention is therefore achieved by an active ingredient-peptide construct, comprising an active ingredient A and a peptide B, wherein the construct has a net charge that is negative at pH 6 and wherein the active ingredient-peptide construct is free from a constituent C which can pass through the membrane of a biological cell.

By an active ingredient-peptide construct is meant within the framework of the invention any molecule which comprises an active ingredient A which is bonded to at least one peptide B. The active ingredient A and peptide B can thus be bonded in any manner known to a person skilled in the art to be suitable. Preferably the active ingredient A and the peptide B of the active ingredient-peptide construct according to the invention are covalently directly bonded to one another, but it is also preferred that the active ingredient A and the peptide B are bonded to one another via a linker.

A molecule as linker is characterized in that, due to its nature, it makes no, or only an insubstantial, contribution to the function of the total molecule formed by the linker, and serves only to bring to a desired length the distance between a first part X of a total molecule which has one property, and another part Y of a total molecule which has the same property as X or a different one. E.g. oligomers can be formed with aryl acetylene, diamines (e.g. ethylenediamine or diaminopropane), polyfunctional acids, polyethylene oxide, polypropylene oxide, amino acids, amino acid derivatives or ethylene glycol which contain 2 to 10 of these monomer units or combinations thereof. Linkers can also be constructed from branched or unbranched alkanes,

Examples of suitable reactions for the production of linkers are e.g. the formation of amides from carboxylic acids and amines, of secondary and/or tertiary amines from haloaliphates and amines, addition to double bonds, the formation of ethers or thioethers from halo-carboxylic acids and thiols, of thioethers from thiols and maleinimides, of amide bonds from thioesters and 1,2-aminothiols, of thioamide bonds from dithioesters and 1,2-aminothiols, of thiazolidines from aldehydes and 1,2-aminothiols, of oxazolidines from aldehydes/ketones and 1,2-aminoalcohols, of imidazoles from aldehydes/ketones and 1,2-diamines, (such as eag. used in FIG. 3), of thiazoles from thioamides and alpha-halo-ketones, of aminothiazoles from amino-oxy-compounds and alpha-isothiocyanato-ketones, of oximes from amino-oxy-compounds and aldehydes, of oximes from amino-oxy-compounds and ketones, of hydrazones from hydrazines and aldehydes, of hydrazones from hydrazides and ketones and numerous others.

Within the framework of the present invention methods are particularly preferred which produce linkers with an internuclear distance of between 4 and 40.

If the construct according to the invention comprises a linker, it is preferred if the linker has a chain of 4 to 40 C—C bonds.

The active ingredient-peptide construct can be both a combination of one or more active ingredients A of the same or different type with one or more peptides B of the same or different type. Furthermore, in this case the peptide(s) B and the active ingredient(s) A can be bonded in the same or different manner. By ‘active ingredient-peptide construct” is thus also meant a construct of one or more active ingredients which can be subsumed under the term “active ingredient A” and one or more peptides B which can be subsumed under the term “peptide B”.

The active ingredient-peptide construct of the invention must further have a negative net charge at pH 6 in order to achieve the object according to the invention. The net charge of a peptide sequence can as a first approximation be determined by a calculation, as stated e.g. in WO 2003/097706, or more precisely and according to the invention by corresponding biochemical experiments such as e.g. isoelectric focusing (Lexikon der Biochemie) or titration (according to Fresenius' Journal of Analytical Chemistry 274(1975)359-361) or ascertained according to Helvetica Chimica Acta. 91(2008)468-482).

It is known to a person skilled in the art that the net charge of a molecule results from the sum of the part-charges of the individual functional groups.

The inventors of the present application surprisingly found that—in order to guarantee that the construct according to the invention remains in the extracellular space—the construct also must not contain a constituent C which can pass through the membrane of a biological cell. By a “constituent C” is meant within the framework of the invention an additional constituent which differs from the constituents A (active ingredient) and B (peptide) of the construct and is also not part of the constituents A or B. An additional constituent C can be a peptide with more than one positively charged, i.e. basic, amino acid which can pass through the membrane of a biological cell. The constituent C can for example comprise one or more different amino acids selected from the group consisting of arginine, lysine and histidine. By a constituent C can however also be meant for example any other molecule which is different from the constituents A (active ingredient) and B (peptide) of the construct and is also not part of the components A or B and which can pass through the membrane of a biological cell.

It is preferred that the constituent C is a peptide with more than two amino acids, selected from the group consisting of lysine, arginine and histidine, preferably more than three amino acids, preferably more than four amino acids, more preferably more than five amino acids, even more preferably more than six amino acids.

According to a preferred embodiment of the active ingredient-peptide construct according to the invention the peptide B bonded to the active ingredient A is constructed from 2 to 70 amino acids, preferably 2 to 50 amino acids, further preferably 2 to 30 amino acids and particularly preferably 2 to 25 amino acids. Where the construct according to the invention comprises more than one peptide B, these can be constructed from the same number or a different number of amino acids.

Amino acids are organic acids which contain at least one and usually not more than four amino groups NH₂ and at least one and usually not more than 4 carboxyl groups. Depending on the position of the amino group in the carbon chain relative to the terminal carboxyl group COOH a distinction is drawn between alpha, beta, gamma amino acids etc. (Lexikon der Biochemie). By amino acids are meant within the framework of the present application all amino acids according to the above definition irrespective of the occurring chirality. Also meant are amino acids which have more than one centre of chirality with different topological properties which are thereby possible.

Basic amino acids have a side chain with a positive charge at pH 6, such as e.g. arginine, lysine, histidine or other amino acids which have these properties.

An acid amino acid has a side chain with a negative charge at pH 6, such as glutamic acid, aspartic acid, phosphoserine, phosphothreonine or other amino acids which have this property.

The peptide B of the active ingredient-peptide construct according to the invention can be composed of all the amino acids which are known to a person skilled in the art to be suitable for the purpose according to the invention and which bring about an active ingredient-peptide construct with a net charge which is negative at pH 6. In a particularly preferred embodiment the peptide B of the active ingredient-peptide construct according to the invention is composed of amino acids which are selected from the group consisting of glutamic acid, aspartic acid, phosphoserine or phosphothreonine. Where the construct according to the invention comprises more than one peptide B, these can be constructed from the same or different amino acids.

In a particularly preferred embodiment the peptide B of the active ingredient-peptide construct according to the invention has an amino acid sequence selected from the group consisting of (Glu)₄, (Glu)₅, (Glu)₆, (Glu)₇, (Asp)₄, (Asp)₅, (Asp) ₆, (Asp)₇ or sequences of the lengths 4-7 which contain Asp and Glu, irrespective of the precise order of these amino acids.

Amino acids which form peptides which are degradable with difficulty, or not at all, in the extracellular space, such as e.g. D-amino acids, are particularly suitable.

Any active ingredient A known to a person skilled in the art to be suitable for the purpose according to the invention can be used as active ingredient A of the active ingredient-peptide construct according to the invention. Active ingredients which are supposed to and/or can develop their effect only outside a biological cell are suitable in particular. By this are meant within the framework of the invention on the one hand active ingredients which develop a toxic effect or at least an undesired (side-)effect within biological cells. Also meant by this are on the other hand, however, active ingredients which do not have an effect inside a biological cell and whose concentration must be compensated for outside of the cell by administration of increased doses because of the migration into the cell. Finally, active ingredients which can be better administered by bonding with suitable peptides, for example not just because they can be guaranteed to remain in the extracellular space, but their solubility can be improved, are particularly suitable.

In a preferred embodiment, the active ingredient A of the active ingredient-peptide construct according to the invention is a pharmaceutical active ingredient.

Corresponding to a further preferred embodiment of the construct according to the invention it is provided that the active ingredient A is an active ingredient which has a positive net charge at pH 6.

Preferred active ingredients A of the active ingredient-peptide construct of the invention are effectors which can inhibit inflammatory processes in biological objects, preferably in veterinary and human medicine. Effectors of peptidyl-prolyl cis/trans isomerases (PPIases) are particularly preferred. In a further particularly preferred embodiment the effectors comprise substances which inhibit the enzymatic activity of cyclophiline, wherein it is in particular preferred that by inhibition is meant that the reduction of the catalytic activity caused by the active ingredient under optimal conditions is at least 50%.

Effectors bring about a number of effects which can be used therapeutically. Thus they can for example have an influence on immunosuppression, neuroprotection/neurogeneration, chaperone activity, HIV infection, cancer or Alzheimer's.

Examples of effectors preferred according to the invention are PPIase inhibitors. While these effectors can differ between the individual PPIase families (Nature Chemical Biology. 3(10):619-29, 2007; Cellular & Molecular Life Sciences. 63(24):2889-900, 2006; Current Topics in Medicinal Chemistry, 3(12):1315-47, 2003; Advances in Protein Chemistry. 59:243-82, 2001), they often have similar inhibiting power compared with sequence-similar family members, Because PPIases within a family can influence very different biochemical reactions, the diagnostic or pharmacological effect of administered active ingredients depends directly on the concentration reached in very different distribution spaces. Thus e.g. some of these PPIase inhibitors (e.g. Biopolymers 84(2006)125-146; Chemical & Pharmaceutical Bulletin. 54(3):372-376, 2006; Chemistry & Biology. 10(1):15-24, 2003; Nucleic Acids Research. 29(3):767-773, 2001) such as e.g. the therapeutically used cyclosporin can be dissolved in water only with difficulty (DE 19859910).

In spite of this, according to customary medicinal-product solvent application, much higher concentrations are found within cells. It is presumed that the active ingredients pass through the membrane of a biological cell and then bond to PPIases intracellularly present. For a cyciosporin derivative (SDZ IMM 125) thus (Anti-Cancer Drugs. 8(4):400-404, 1997; Journal of Pharmacokinetics & Biopharmaceutics. 22(5):327-65, 1994) an approximately 8× higher concentration was detected in blood cells than in the surrounding plasma.

The effectors (active ingredient A) preferred within the framework of the invention include:
a) EZ's osteoporosis-influencing polypeptides (such as IGFIIE, IGFBP2, presented in detail in U.S. Pat. No. 6,916,790).
b) CS1 peptides and their fragments which can influence the adherence of lymphocytes in a therapeutically desired manner, as comprehensively described in U.S. Pat. No. 7,238,668.
c) Inhibitors acting on TGF-beta, such as e.g., NAALADase inhibitors which regulate TGF-beta and act on very different diseases such as e.g. neurodegenerative diseases, “extracellular matrix formation disorders”, illnesses related to cell growth, infectious diseases, diseases of the immune system, “epithelial tissue scarring”, “collagen vascular diseases“, “fibroproliferative disorders”, “connective tissue disorders”, inflammations, air-passage syndrome or infertility as is shown e.g. in U.S. Pat. No. 6,444,657, but also compounds which, as described in U.S. Pat. No. 6,693,118, have a therapeutically useful effect on the extracellular TGF-beta concentration.
d) Cytokines such as oncostatin-M or its biologically active fragments or protein constructs which have a similar effect on tumour cells, as is shown in summary e.g. in U.S. Pat. No. 5,744,442.
e) Spirocyclic-6,7-dihydro-5H-pyrazolo[1,2-a]pyrazol-1-ones which act on inflammatory cytokines, as is shown e.g. in U.S. Pat. No. 6,566,357, U.S. Pat. No. 6,821,971 or U.S. Pat. No. 6,730,668.
f) 1,1,3-tri-substituted urea compounds which can act on inflammatory cytokines, as is shown e.g. in U.S. Pat. No. 7,449,474.
g) Substituted pyrrolo[3,2-d]pyrimidine-2,4-diones which act as adenosine-receptor antagonists and can likewise act on inflammatory cytokines, as is shown e.g. in U.S. Pat. No. 7,449,473.
h) Compounds which influence the effect of secreted TNFalpha, such as etanercept (Enbrel), inflixamab (Remicade), as is shown e.g. in U.S. Pat. No. 6,881,407.
i) Compounds which are radioactively or paramagnetically labelled in order to be able to recognize primary tumours or metastases, as is stated e.g. in U.S. Pat. No. 5,733,892 or U.S. Pat. No. 7,329,644.
j) Compounds which are used for chemotherapy in order to suppress the growth of tumours and their metastases, as is shown in U.S. Pat. No. 7,148,196.
k) Taurolidine and its medicinally active derivatives which can influence the growth of tumours and metastases, as is shown e.g. in U.S. Pat. No. 7,122,541.
l) Crown ether compounds which are suitable for bonding to viruses, as is described e.g. in U.S. Pat. No. 5,314,878.

m) Compounds with which viruses can be inactivated, as is shown e.g. in U.S. Pat. No. 5,120,649.

PPIase inhibitors which are particularly preferably used as effectors (active ingredient A) within the framework of the invention are:
Pin1 inhibitors, which can be antibodies, antisense oligonucleotides or even short nucleic acid molecules (siRNA) or small organic compounds such as e.g. juglone or aromatic structures such as PIA, PIB, PIC, PID, PIE, PIE, PIJ, as described in U.S. Pat. No. 7,417,072 or Biopolymers 84(2006)125-146.
These inhibitors can e.g. inhibit the PPIase activity of extracellular Pin1 and thus have a curative effect on faulty immune system controls which e.g. accompanies the increased number of eosin-positive cells in the blood. Pin1 inhibitors can, however, also therapeutically influence the disease-connected expression of cytokines, as is observed for example in asthmatic diseases or else also with the undesired rejection of transplanted organs or act as anticarcinogens or antifungals (e.g.: Cellular & Molecular Life Sciences 65(2008)359-375), Some part of the Pin1 effect can possibly be traced back to the influencing of the TGF1-beta concentration (e.g.: Journal of Clinical Investigation 118(2008)479-490; Journal of Allergy & Clinical Immunology 120(2007)1082-1088).
Peptide mimetics of the phospho-Ser-Pro or phospho-Thr-Pro motif.
Peptides as described by Wang at al, (JACS 126(2004)15533-155542; Biopolymers 84(2006)125-146) including their antiproliferative effect.
Spiroannulated 3-benzofuranones (Molecules. 13(2008)995-1003).
Sulphonamides of heterocyclic thioesters (U.S. Pat. No. 7,410,995, U.S. Pat. No. 7,265,150, U.S. Pat. No. 7,338,976).
Neurotrophic N-glyoxyl-prolyl esters (U.S. Pat. No. 7,282,510, U.S. Pat. No. 5,859,031).
Heterocyclic compounds (U.S. Pat. No. 6,562,964 and U.S. Pat. No. 6,372,736).
General FKBP inhibitors (U.S. Pat. No. 6,291,510 and U.S. Pat. No. 6,140,357, U.S. Pat. No. 6,509,477, U.S. Pat. No. 6,509,477, U.S. Pat. No.6,509,477).
FKBP-bonding pipecolic acid derivatives (U.S. Pat. No. 6,500,843, U.S. Pat. No. 6,022,878, U.S. Pat. No. 5,846,981, U.S. Pat. No. 5,843,960, U.S. Pat. No. 5,801,197) PPIase inhibitors.
FKBP inhibitors U.S. Pat. No. 6,509,464, U.S. Pat. No. 6,495,549, U.S. Pat. No. 6,166,0111.
FKBP inhibitors are currently predominantly used or proposed for use as active ingredients with neurotrophic effects and are suitable for the treatment of nervous disorders (e.g.:WO 96/40140, WO 96/40633, PNAS 91(1994)3191-95, Nature Medicine 1(1995) 32-37, WO 96/40140, WO 96/40633, WO 97/16190, U.S. Pat. No. 7276498) possibly caused by inhibition of FKBP-12 or FKBP-52, as an active ingredient with immunosuppressive properties for the prevention of transplant rejection (Clinical Chemistry 39(1093)2219-2228, Current Opinion in immunology 5(1993) 763-773, Transplantation Proceedings 38(2006)1823-1824), for therapeutic influencing of benign and malignant tumours (e.g.: Current Problems in Cancer (2008)161-177, Molecular Cancer Therapeutics 7(2008) 1347-1354, Cancer 100(2004)657-666), for the treatment of diseases associated with inflammations (Transplantation Proceedings 37(2005)1880-1884, Molecular Pharmacology 65(2004)880-889, Journal of Biological Chemistry 278(2003)45117-45127, Inflammation Research 49(2000)20-26) or for influencing angiogenesis (e.g.: Hepatology Research 38(2008)1130-1139, WEBS Letters 582(20008)3097-3102, Clinical Cancer Research 13(13):3977-3988, 2007).
Heteroaryl-pyrrolidine, piperidine and homopiperidine derivatives (U.S. Pat. No. 6,686,357).
FK506 conjugates with amyloid-bonding peptides for the treatment of neurological diseases such as Alzheimer's, multiple sclerosis or amyotrophic lateral sclerosis (U.S. Pat. No. 6,316,405).
Tumour antigen peptides and corresponding derivatives derived from cyclophilin B or cyclophilin (U.S. Pat. No. 7,368,107; U.S. Pat. No. 7,041,297).
Non-peptide compounds suitable for being able to therapeutically influence the regeneration of neuronal cells (U.S. Pat. No. 6,677,376).
Therapeutically usable cyclic hydrocarbons (U.S. Pat. No. 6,656,971).
Compounds which can therapeutically influence alopecia, parasite attack but also HIV virus infections (U.S. Pat. No. 6,593,362).

Non-immunosuppressive 6-position cyclosporin analogues (U.S. Pat. No. 4,941,88).
Cyclosporin analogues for therapeutically influencing the immune and respiratory systems (U.S. Pat. No. 7,226,906, U.S. Pat. No. 7,141,648, U.S. Pat. No. 6,927,208, U.S. Pat. No. 5,994,299; U.S. Pat. No. 5,977,067, U.S. Pat. No. 5,965,527, U.S. Pat. No. 4,288,431, U.S. Pat. No. 6,455,518, U.S. Pat. No. 6,432,968, U.S. Pat. No. 6,046,328).
Cyclosporin alkines (U.S. Pat. No. 7,378,391, U.S. Pat. No. 7,361,636).
Deuterated cyclosporin analogues (U.S. Pat. No. 7,358,229), particularly suitable for the treatment of diseases of the immune system.

3-ethers and 3-thioethers of cyclosporin, particularly suitable for the treatment of hepatitis C infections (U.S. Pat. No. 7,196,161).
3-position cyclosporin derivatives, particularly suitable for promoting hair growth (U.S. Pat. No. 6,987,090, U.S. Pat. No. 6,790,830, U.S. Pat. No. 6,762,164).
Cyclosporin analogues, particularly suitable for the treatment of autoimmune diseases (U.S. Pat. No. 6,809,077).
Cyclosporin derivatives, particularly suitable for the treatment of rheumatoid arthritis (U.S. Pat. No. 6,770,279).
Cyclosporin conjugates with amyloid-bonding peptides for the treatment of neurological diseases such as Alzheimer's, multiple sclerosis or amyotrophic lateral sclerosis (U.S. Pat. No. 6,316,405).
Cyclosporin derivatives, particularly suitable for the treatment of HIV infections (U.S. Pat. No. 5,948,884).
8-position cyclosporin derivatives (U.S. Pat. No. 5,318,901).
Cyclosporin peptolides (U.S. Pat. No. 5,116,816), particularly suitable as immunosuppressive, anti-inflammatory and anti-parasitic active ingredients.
6-position cyclosporin analogues with non-immunosuppressive properties (U.S. Pat. No. 4,914,188).
Pharmaceutical compositions for the treatment of transplant rejection, autoimmune or inflammatory diseases using cyclosporin A and 40-O-(2-hydroxyethyl)-rapamycin.
Cyclosporin analogues with modified C-9 amino acids (U.S. Pat. No. 4,885,276; U.S. Pat. No. 4,798,823) which have immunosuppressive properties.

Geldanamycin and its derivatives (U.S. Pat. No. 7,378,407, U.S. Pat. No. 7,259,156, U.S. Pat. No. 6,890,917, U.S. Pat. No. 4,261,989) as antitumour medicinal products.

Fredericamycin and its derivatives (U.S. Pat. No. 7,244,741, U.S. Pat. No. 5,166,208, U.S. Pat. No. 4,673,678) acting as antitumour medicinal products and as antibacterials.
Rapamycin derivatives (rapamycin and its derivatives for the treatment of neurological diseases and as neuroprotective and neuroregenerative substances (U.S. Pat. No. 7,273,874, U.S. Pat. No. 7,282,505, U.S. Pat. No. 7,232,86, U.S. Pat. No. 7,135,298, U.S. Pat. No. 7,045,508, U.S. Pat. No. 7,034,037, U.S. Pat. No. 6,890,546, U.S. Pat. No. 6,808,536, U.S. Pat. No. 6,713,607, U.S. Pat. No. 6,70,9873, U.S. Pat. No. 6,585,764, U.S. Pat. No. 6,432,968, U.S. Pat. No. 6,277,983, U.S. Pat. No. 6,200,985, U.S. Pat. No. 6,200,985, U.S. Pat. No. 6,046,328, U.S. Pat. No. 6,015,809, U.S. Pat. No. 5,989,591, U.S. Pat. No. 5,985,890 U.S. Pat. No. 5,985,325, U.S. Pat. No. 5,955,457, U.S. Pat. No. 5,912,253, U.S. Pat. No. 5,780,462, U.S. Pat. No. 5,776,943, U.S. Pat. No. 5,728,710, U.S. Pat. No. 5,712,129, U.S. Pat. No. 5,661,156, U.S. Pat. No. 5,648,361, U.S. Pat. No. 5,646,160, U.S. Pat. No. 5,491,229, U.S. Pat. No. 5,387,680, U.S. Pat. No. 5,432,183, U.S. Pat. No. 5,362,735, U.S. Pat. No. 5,202,332, U.S. Pat. No. 5,023,262) and in the form of 42-O-alkoxyalkyl derivatives (U.S. Pat. No. 7,217,286), alkylated compounds (U.S. Pat. No. 7,193,078, U.S. Pat. No. 5,922,730, U.S. Pat. No. 5,665,772), carbohydrates (U.S. Pat. No. 7,160,867), deuterated derivatives (U.S. Pat. No. 6,939,878, U.S. Pat. No. 6,884,429, U.S. Pat. No. 6,710,053, U.S. Pat. No. 6,503,921, U.S. Pat. No. 6,342,507), dialdehydes (U.S. Pat. No. 6,680,330), cools (U.S. Pat. No. 6,677,357), conjugates (U.S. Pat. No. 6,541,612, U.S. Pat. No. 6,328,970), 40-O-(2-hydroxyethyl) derivatives (U.S. Pat. No. 6,455,518, U.S. Pat. No. 6,239,124), O-alkylated compounds (U.S. Pat. No. 6,440,990), esters (U.S. Pat. No. 6,432,973), tetrazoles (U.S. Pat. No. 6,329,386, U.S. Pat. No. 6,015,815), oximes, hydroxylamines and hydrazones (U.S. Pat. No. 5,455,249, U.S. Pat. No. 5,446,048, U.S. Pat. No. 5,378,836, U.S. Pat. No. 5,373,014, U.S. Pat. No. 5,455,249, U.S. Pat. No. 5,446,048, U.S. Pat. No. 5,378,836, U.S. Pat. No. 5,373,014, U.S. Pat. No. 5,677,295, U.S. Pat. No. 5,563,145, U.S. Pat. No. 5,023,264), carbamates (U.S. Pat. No. 5,559,120, U.S. Pat. No. 5,567,709, U.S. Pat. No. 5,559,119, U.S. Pat. No. 5,559,112, U.S. Pat. No. 5,550,133, U.S. Pat. No. 5,541,192, U.S. Pat. No. 5,541,191, U.S. Pat. No. 5,637,590, U.S. Pat. No. 5,532,355, U.S. Pat. No. 5,530,121, U.S. Pat. No. 5,530,007, U.S. Pat. No. 5,519,031, U.S. Pat. No. 5,516,780, U.S. Pat. No. 5,508,399, U.S. Pat. No. 5,508,286, U.S. Pat. No. 5,504,204, U.S. Pat. No. 5,489,680, U.S. Pat. No. 5,489,595, U.S. Pat. No. 5,488,054, U.S. Pat. Nos. 5,486,524, 5,486,523, U.S. Pat. No. 5,486,522, U.S. Pat. No. 5,484,791, U.S. Pat. No. 5,484,790, U.S. Pat. No. 5,480,989, U.S. Pat. No. 5,480,988, U.S. Pat. No. 5,463,048, U.S. Pat. No. 5,455,249, U.S. Pat. No. 5,434,260, U.S. Pat. No. 5,411,967, U.S. Pat. No. 5,391,730, U.S. Pat. No. 5,302,584, U.S. Pat. No. 5,262,424, U.S. Pat. No. 5,262,423, U.S. Pat. No. 5,194,447, U.S. Pat. No. 5,118,678), N-oxide esters (U.S. Pat. No. 5,521,194, U.S. Pat. No. 5,559,122, U.S. Pat. No. 5,508,290, U.S. Pat. No. 5,508,285, U.S. Pat. No. 5,491,231), esters (U.S. Pat. No. 5,504,091, U.S. Pat. No. 5,389,639, U.S. Pat. No. 5,416,086, U.S. Pat. No. 5,385,910, U.S. Pat. No. 5,385,909, U.S. Pat. No. 5,385,908, U.S. Pat. No. 5,378,696, U.S. Pat. No. 5,362,718, U.S. Pat. No. 5,358,944, U.S. Pat. No. 5,349,060, U.S. Pat. No. 5,260,300, U.S. Pat. No. 5,233,036, U.S. Pat. No. 5,221,670, U.S. Pat. No. 5,162,333, U.S. Pat. No. 5,130,307, U.S. Pat. No. 5,118,677, U.S. Pat. No. 5,100,883, sulphonates and sulphamates (U.S. Pat. No. 5,346,893, U.S. Pat. No. 5,260,299, U.S. Pat. No. 5,177,203), oxepanes (U.S. Pat. No. 5,344,833, U.S. Pat. No. 5,221,740), imidazolyl derivatives (U.S. Pat. No. 5,310,903), pyrazoles (U.S. Pat. No. 5,169,851, U.S. Pat. No. 5,164,399), acetals (U.S. Pat. No. 5,151,413), ethers (U.S. Pat. No. 5,120,842), dimers (U.S. Pat. No. 5,120,727) or hydrazones (U.S. Pat. No. 5,120,726)

In a preferred embodiment the active ingredient A of the active ingredient-peptide construct of the present invention is selected from the group consisting of cyclosporin A, FK506 and rapamycin.

In a further preferred embodiment the active ingredient A of the active ingredient-peptide construct according to the invention is an active ingredient which is difficult to dissolve and in a particularly preferred embodiment the active ingredient A is an active ingredient which is difficult to dissolve in the extracellular space.

By “active ingredient which is difficult to dissolve” used according to the invention is meant here a pharmaceutically effective substance which at a temperature of 20° C. has a solubility in water of less than 1 g (active ingredient) per 30 ml (water)

In a further particularly preferred embodiment the active ingredient A is cyclosporin A.

Cyclosporin (also ciclosporin) is a cyclic oligopeptide with an immunosuppressive and calcineurin-inhibiting effect. It is characterized by a selective and reversible immunosuppression mechanism. It selectively blocks the activation of T-lymphocytes via the production of specific cytokines which participate in the regulation of these T-cells. Above all the synthesis of interleukin-2 is inhibited, whereby simultaneously the proliferation of cytotoxic T-lymphocytes which e.g. are responsible for the rejection of foreign tissue is suppressed. Cyclosporin acts intracellularly by binding to the so-called cyclophilines or immunophilines which belong to the family of cyclosporin-binding proteins.

Cyclophilin inhibitors have a very broad therapeutic range, such as e.g. the treatment of diseases of the respiratory tract, such as e.g. asthma, COPD, pulmonary inflammation or emphysema (Expert Opinion on Investigational Drugs 12(2003)647-653, Biodrugs 8(1997) 205-215, American Journal of Respiratory Cell & Molecular Biology 20(1999)481-492), metabolic diseases such as diabetes (Transplantation Proceedings 37(2005)1857-1860, Molecular Pharmacology 60(2001)873-879), inflammatory diseases of the digestive tract (Bone Marrow Transplantation 26(2000):545-551, Pharmaceutical Research 20(2003)910-917), disorders of the immune system (Immunology Letters 84(2002)137-143, Acta Biochimica Polonica 49(2002)233-247, inflammations (Journal of Periodontal Research 42(2007)580-588, Journal of Neurology, Neurosurgery & Psychiatry 76(2005)1115-1120, Transplant Immunology 12(2004):151-157), cardiovascular diseases (Journal of Hypertension 17(1999)1707-1713, Drug & Chemical Toxicology 21(1998)27-34), neurological diseases (Annals of Vascular

Surgery. 20(2006) 243-249), diseases which involve disturbance of angiogenesis (Blood Purification. 25(2007)466-472, International Angiology 24(2005)372-379, Nefrologia. 23(2003):44-48), to suppress the immune response in organ transplantation (Bone Marrow Transplantation. 38(2006)169-174), Biodrugs. 14(2000)185-193, Clinical Immunotherapeutics. 5(1996)351-373) and autoimmune diseases (Immunology & Immunopathology. 82(33:197-202, 1997), arthritic diseases (British Journal of Rheumatology. 36(1997)808-811, Biodrugs. 7(1997)376-385), dermatitides (Veterinary Dermatology 17(2006)3-16), psoriasis (Journal of Dermatological Treatment 16(2005)258-277, Hautarzt 44(1993)353-360), in allergies (Cornea 27(2008)625, Journal of Small Animal Practice 47(2006)434-438, Clinical & Experimental Ophthalmology 34(2006)347-353), in multiple sclerosis (Immunopharmacology & Immunotoxicology 21(1999)527-549, Journal of Neuroimaging 7(1997)1-7), diseases which have been caused by ischemia, such as e.g. infarctions of the heart (Annals of Thoracic Surgery 86(2008)1286-1292, Acta Anaesthesiologica Scandinavica 51(2007)÷909-913), of the pancreas (Pancreas 32(2006)145-151) or of the brain (Annals of Vascular Surgery 20(2006)243-249, Neurological Research 27(2005)827-834), kidney diseases such as e,g. glomerulonephritis (Nephrology Dialysis Transplantation 19(2004)3207, Nephron 91(2002)509-511), tumours (Journal of Investigative Dermatology 128(2008)2467-2473, Endocrinology 148(2007)4716-4726), in multiple myelomas (Leukemia 12(1998)505-509, Leukemia & Lymphoma 16(1994)167-170), in acute or chronic leukaemia (Cancer Chemotherapy & Pharmacology 52(2003)449-452, Cancer 97(2003)1481-1487), muscle degeneration (Neuroscience Research Communications 31(2002)85-92, cachexia (International Journal of Cardiology 85(2002)173-183, Drugs 58(1999)953-963, 1999), Reiter's syndrome (Rheumatology 40(2001)945-947), bone degradation diseases (European Journal of Pharmacology 564(2007)226-231, Biochemical & Biophysical Research Communications 254(1999)248-252), in Alzheimer's disease (Biochemical & Biophysical Research Communications 248(1998)168-173, Chinese Medical Journal 115(2002)884-887), malaria (Molecular & Biochemical Parasitology 99(1999)167-181), septic and toxic shock syndrome (Journal of Pharmacology & Experimental Therapeutics 311(2004)1256-1263), myalgia (British Journal of Dermatology 147(2002)606-607), in viral infections (Expert Opinion on Emerging Drugs 13(2008)393-416) such as e.g. HIV-1, HIV-2, HIV-3 (Journal of Infectious Diseases 194(2006)1677-1685, Molecular Medicine Today 1(1995)287-291, 1995), cytomegaloviruses (Journal of Virology 81(2007)9013-9023) or adenoviruses (Ophthalmologe 105(2008)592-594, Ophthalmologe 97(2000)764-768) and to promote hair growth (Archives of Dermatological Research 296(6):265-269, 2004, Annales de Dermatologie et de Venereologie 127(2000)769).

The complex of cyclosporin and cyclophilin then blocks the serine-threonine-phosphatase calcineurin. Its activity state in turn controls the activation of transcription factors such as, say, NE-Kappa B or NFATp/c which play an important role in the activation of various cytokine genes, also including interleukin-2. The immunocompetent lymphocytes are hereby arrested during the G0 or G1 phase of the cell cycle, because the proteins essential for cell division such as interleukin-2 can no longer be produced. T helper cells which increase the activity of the cytotoxic T cells responsible for rejection are the preferred point of attachment for cyclosporin.

In addition cyclosporin inhibits the synthesis and release of further lymphokines which are responsible for the proliferation of mature cytotoxic T lymphocytes and for further functions of the lymphocytes. The ability of cyclosporin to block interleukin-2 is critical for its clinical effectiveness: Transplant recipients who display a good tolerance of their transplants are characterized by a low production of interleukin-2. Conversely, in patients with a manifest rejection reaction, no inhibition of interleukin-2 production can be established. All the effects above observed under the paragraph “effects of cyclophilin inhibitors” have been described for cyclosporin and its derivatives.

In a further particularly preferred embodiment the active ingredient A is FK506 or rapamycin.

In a further preferred embodiment the peptide B is covalently bonded to the active ingredient A. The peptide can, however, be joined in principle to the active ingredient A in any manner known to a person skilled in the art to be suitable for the purpose according to the invention.

Within the framework of the present invention the active ingredient-peptide construct can comprise further groups which provide the active ingredient-peptide construct with further properties which are desirable for a person skilled in the art for the respective purpose provided it is free from a constituent C as defined above in more detail. The active ingredient-peptide construct according to the invention can be bonded to one, but also to more than one, group which can be either identical, similar or different. The inclusion of additional groups in the active ingredient-peptide construct according to the invention can serve on the one hand to reinforce already-present properties, but on the other hand it is also possible to provide the construct with new, further properties. It is for example conceivable that the construct is provided with an indicator in order to monitor its concentration in the desired tissue or in order to be able to classify the desired tissue by means of indicator distribution. It is further conceivable that the construct is provided with a group which makes possible its concentration in quite specific tissues.

In a preferred embodiment the active ingredient-peptide construct comprises an indicator.

In a further preferred embodiment the indicator is covalently bonded to the active ingredient A. In principle the indicator can, however, be bonded to the active ingredient A in any manner known to a person skilled in the art to be suitable for the purpose according to the invention.

In a further preferred embodiment the indicator is covalently bonded to the peptide B. In principle the indicator can, however, be bonded to the peptide B in any manner known to a person skilled in the art to be suitable for the purpose according to the invention.

In a particularly preferred embodiment the indicator is covalently bonded to a linker which joins the peptide B to the active ingredient A.

The linker can, within the framework of the present invention, be any compound known to a person skilled in the art to be suitable for the purpose according to the invention. Preferably it is, however, a compound which is free from a protease interface. The linker is particularly preferably selected from the group consisting of molecules which form an internuclear distance of between four and 40 atoms.

By “indicator” are meant within the meaning of the invention substances such as preferably dyes, voltage-sensitive indicators, ph indicators, calcium-sensitive indicators, radioactive elements, NMR labels or electron-spin labels which are repeatedly described in the scientific literature (WO/2005/022158, EP 0649022, U.S. Pat. No. 6,596,499, U.S. Pat. No. 7,090,995, U.S. Pat. No. 4,672,044). The term indicator covers, within the meaning of the invention, preferably individual atoms or molecules which are covalently bonded to the construct. An indicator or also more than one indicator can be covalently bonded directly to the active-ingredient molecule, but the indicator or also more than one indicator can also be bonded to a polyfunctional linker or the indicator or also more than one indicator can also be bonded covalently inside the acid peptide or terminally to the acid peptide. The term “indicator” preferably covers dyes, voltage-sensitive indicators, pH-sensitive indicators, radioactive elements, calcium-sensitive indicators, NMR labels and electro-spin labels.

“Dyes” within the meaning of the invention are substances which can be visually detected by detecting the electromagnetic radiation emitted from or not absorbed by them. These include e.g. dyes such as fluorescein isocyanate (FTC), fluorescein isothiocyanate (FITC), dimethylamino naphthalene-S-sulphonyl chloride (DANSC), tetramethylrhodamine isothiocyanate (TRITC), lissamine rhodamine B200 sulphonyl chlorides (RB 200 SC) etc. A description of numerous suitable molecules is e.g. to be found in DeLuca, “Immunofluorescence Analysis”, in “Antibody As A Tool”, Marchalonis et al., Eds., John Wiley & Sons, Ltd., pp. 189-231, (1985).

“Voltage-sensitive indicators” within the meaning of the invention are substances which, depending on an adjacent electric potential difference or the existing electric potential, change their physical, optical or catalytic properties such that these trigger a detectable signal. Voltage-sensitive indicators such as e.g. DIBAC [Japanese Journal of Pharmacology 86(2001)342-350, American Journal of Physiology—Heart & Circulatory Physiology 287(2004)H985-H993) are known to a person skilled in the art.

“pH-sensitive indicators” within the meaning of the invention are substances which, depending on the pH, change their physical, optical or catalytic properties such that these trigger a detectable signal. Such indicator dyes, such as e.g. phenol red, bromothymol blue, bromophenol blue and many others are known to a person skilled in the art.

“Calcium-sensitive indicators” within the meaning of the invention are substances which, in the presence of calcium, change their physical, optical or catalytic properties such that these trigger a detectable signal. Calcium-sensitive indicators known to a person skilled in the art are e.g. aequorin and other calcium-sensitive dyes such as e.g. FURA-2.

“Radioactive elements” within the meaning of the invention produce e.g. gamma radiation, such as e.g. the following isotopes ¹²⁴J, ¹²⁵J, ¹²⁸J, ¹³¹J, ¹³²J or ⁵¹Cr, wherein ¹²⁵J is to be particularly preferred. Others, such as e.g. ¹¹C, ¹⁸F, ¹⁵O or ¹³N, can be detected by means of their positron radiation and corresponding detectors (positron-emission tomography) and others, such as e.g. ¹¹¹In, by means of electron capture.

“NMR labels” within the meaning of the invention are substances in which atoms with an odd number of nucleons (sum of the protons and neutrons) are contained. Such nuclei, e.g.: ¹³O, ¹⁵N or ¹⁹F, have a nuclear spin and thus a nuclear magnetic moment.

“Electron-spin labels” serve, within the meaning of the invention, to measure “electron paramagnetic resonance” by means of electron-spin resonance. The resonant microwave absorption of a sample is measured in an external magnetic field. Thus molecules can be detected which have a permanent magnetic moment (unpaired electrons) (Physics in Medicine & Biology. 43(1998)U 3-U 4, Clinical Chemistry & Laboratory Medicine. 46(2008)1203-1210).

The active ingredient-peptide construct according to the invention can contain one or also more than one indicator which can be identical but also different in nature.

The use of indicators is particularly advantageous if the active ingredient-peptide construct according to the invention is to be used for the production of a medicinal product for use in a therapeutic process such as, for example, a diagnostic procedure (e.g. an anamnesis investigation, physical examination, use of imaging processes such as X-ray/MRT or analysis with laboratory values of blood and other bodily fluids). If the active ingredient-peptide constructs according to the invention also contain one or more indicators, the distribution space of the active ingredient A can be recognized by means of these indicators. Indicators can also be used to quantify the active ingredient A.

In a further preferred embodiment the active ingredient-peptide construct according to the invention is also free from a protease interface, in particular free from a protease interface which splits off the peptide B from the construct after the cut, such as for example a linker of amino acids suitable for this purpose (such as are known to a person skilled in the art), which could, for example, serve to bond together the individual groups of the construct.

If individual groups are not bonded directly, but via a splittable linker, this could, under certain circumstances—for example an unintended side-effect when administering several medicinal products would be conceivable—lead to an unintended cleavage and thus to the loss of the group bonded via the linker in each case.

The active ingredient-peptide construct according to the invention relates in particularly preferred embodiments to constructs or a molecule with the following formulae, in which R, if included, is a carboxy-tamra or acetyl radical.

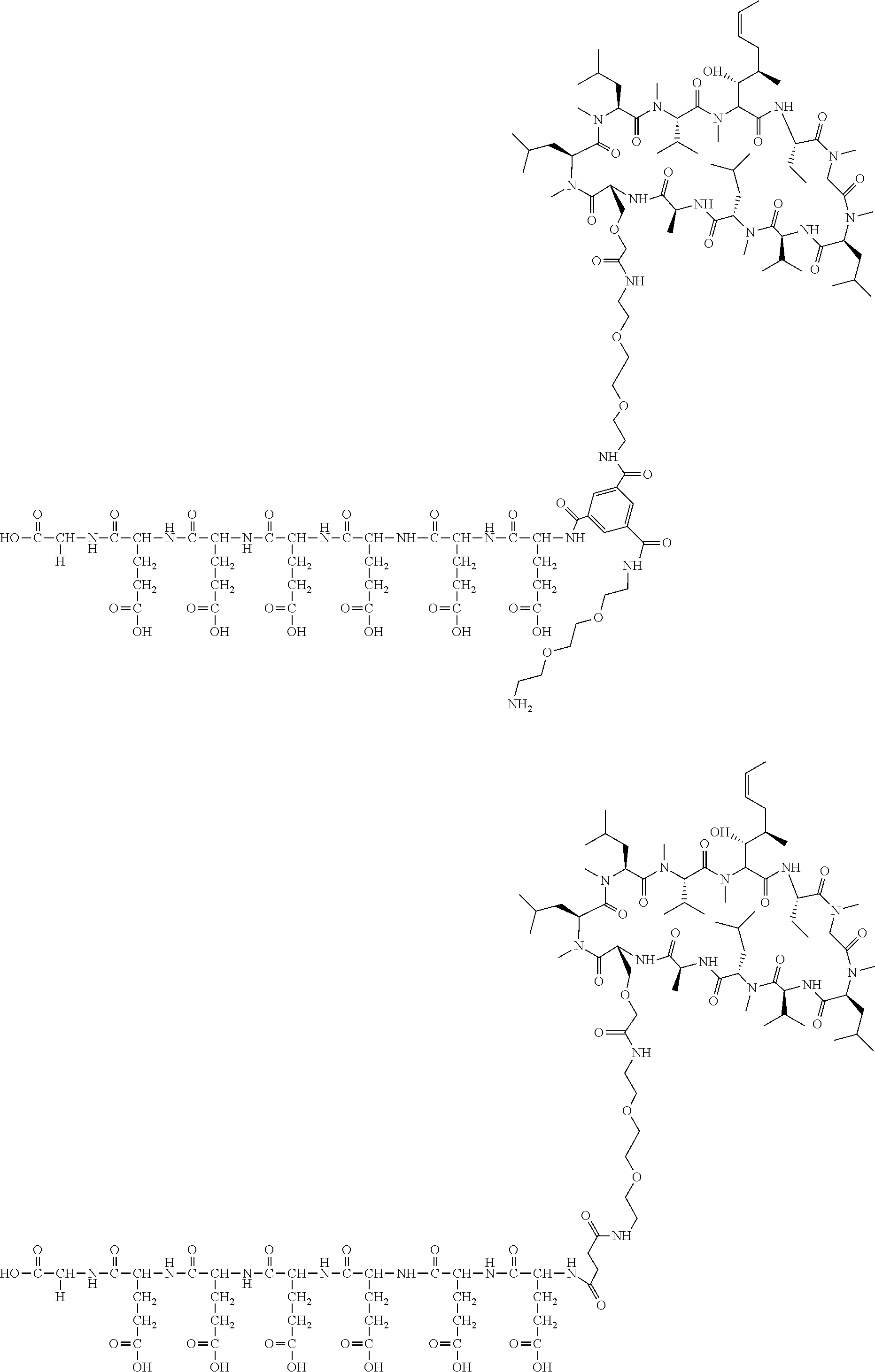

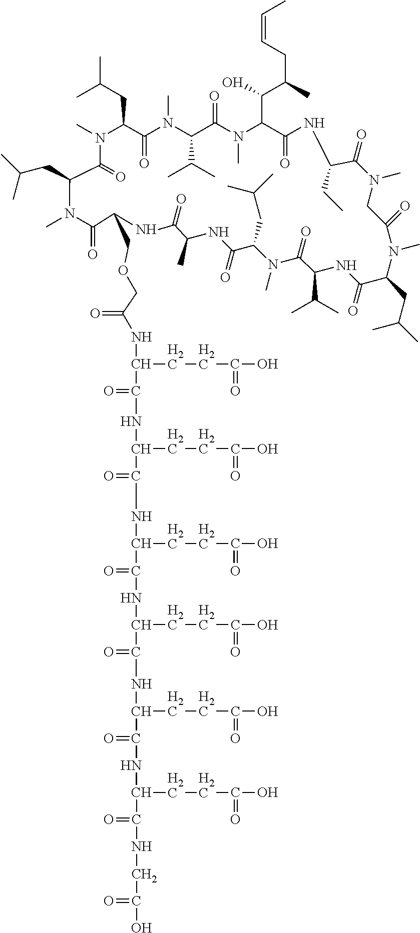

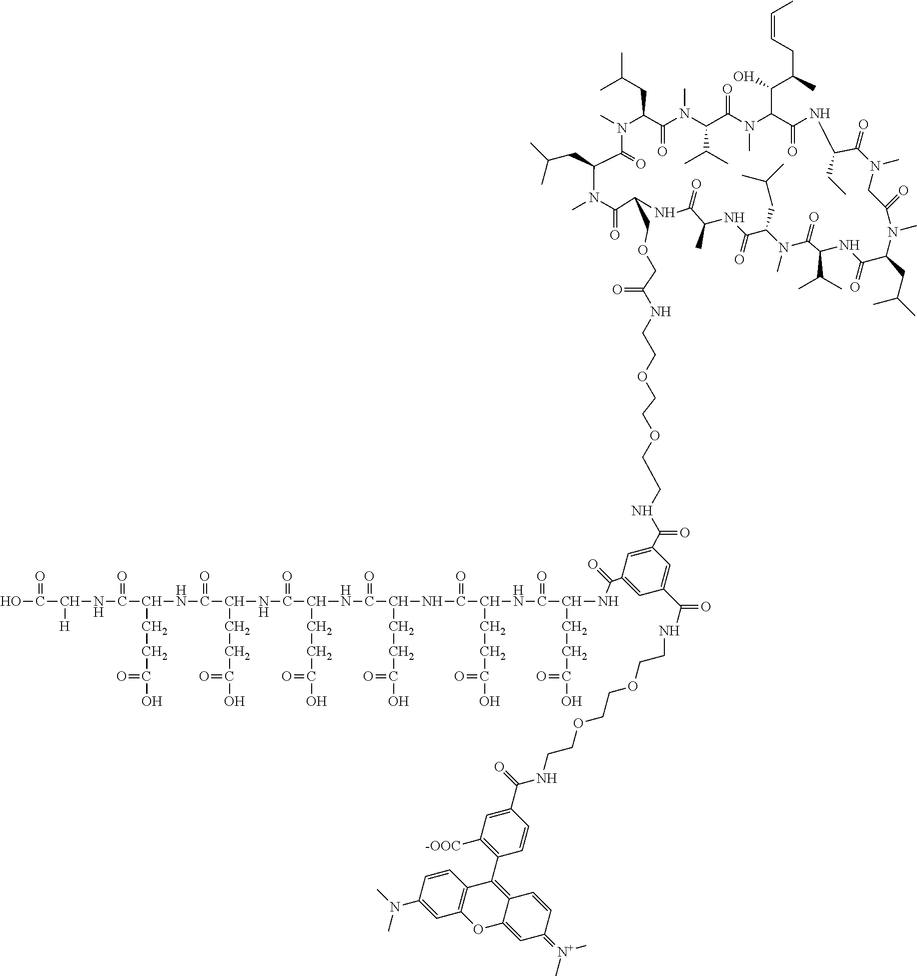

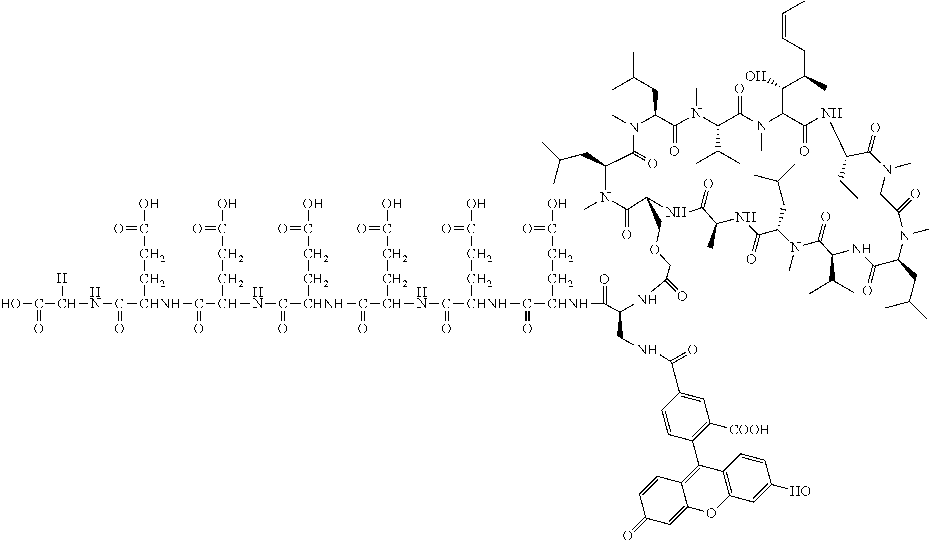

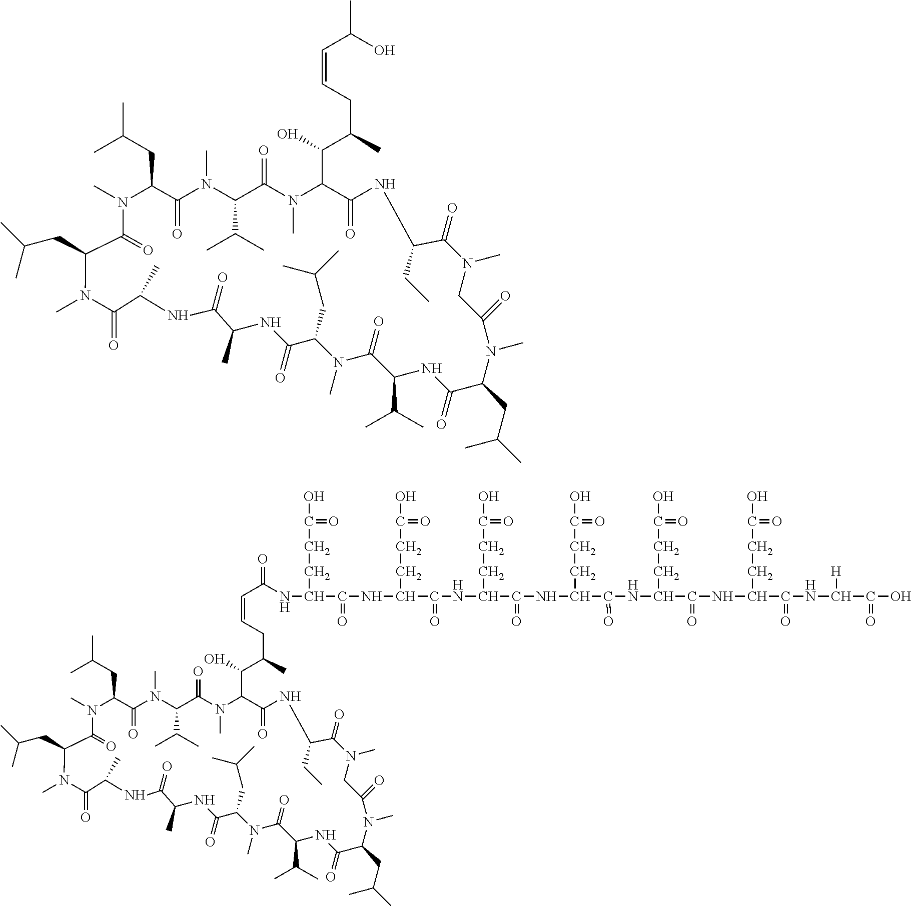

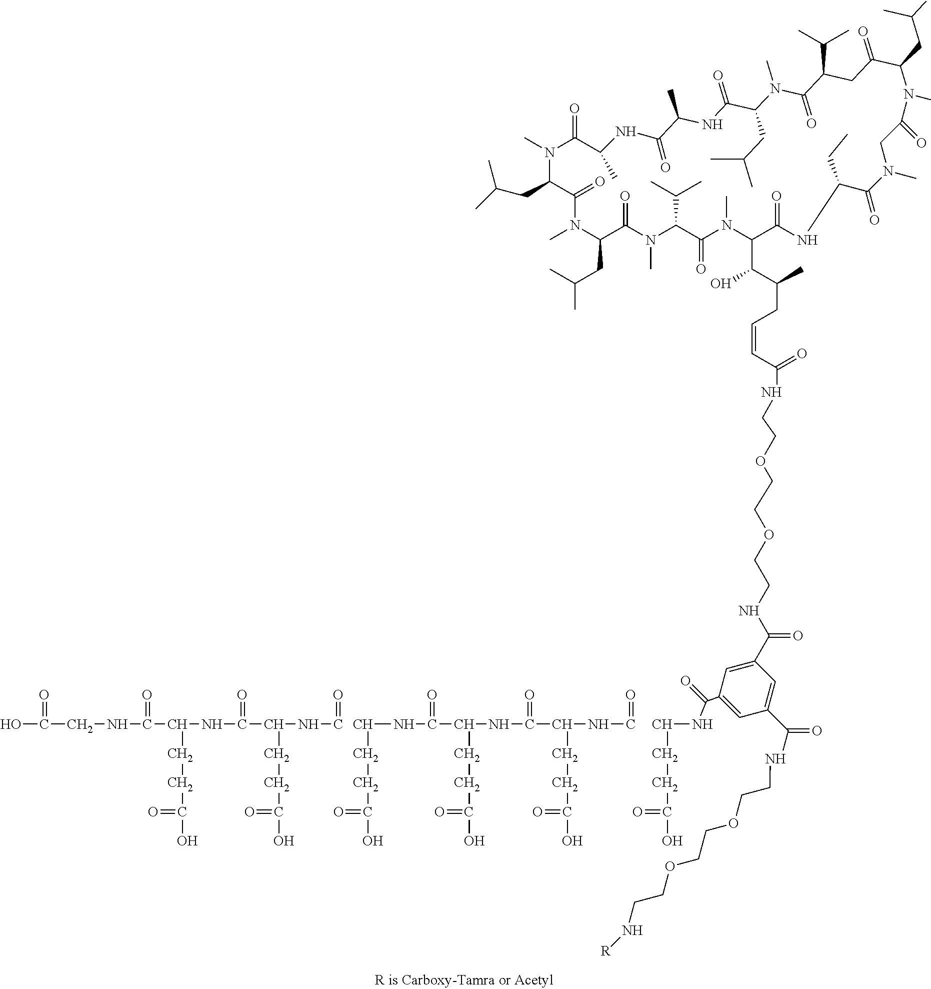

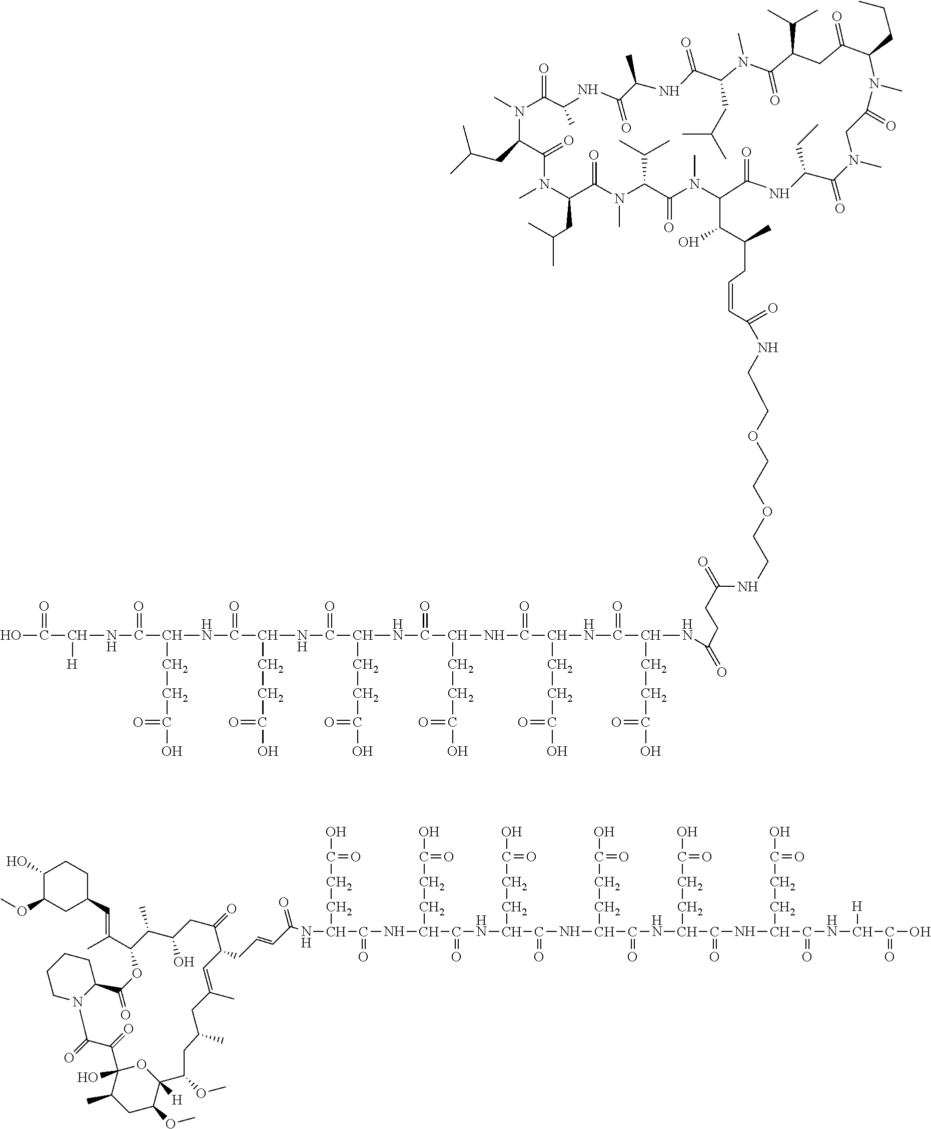

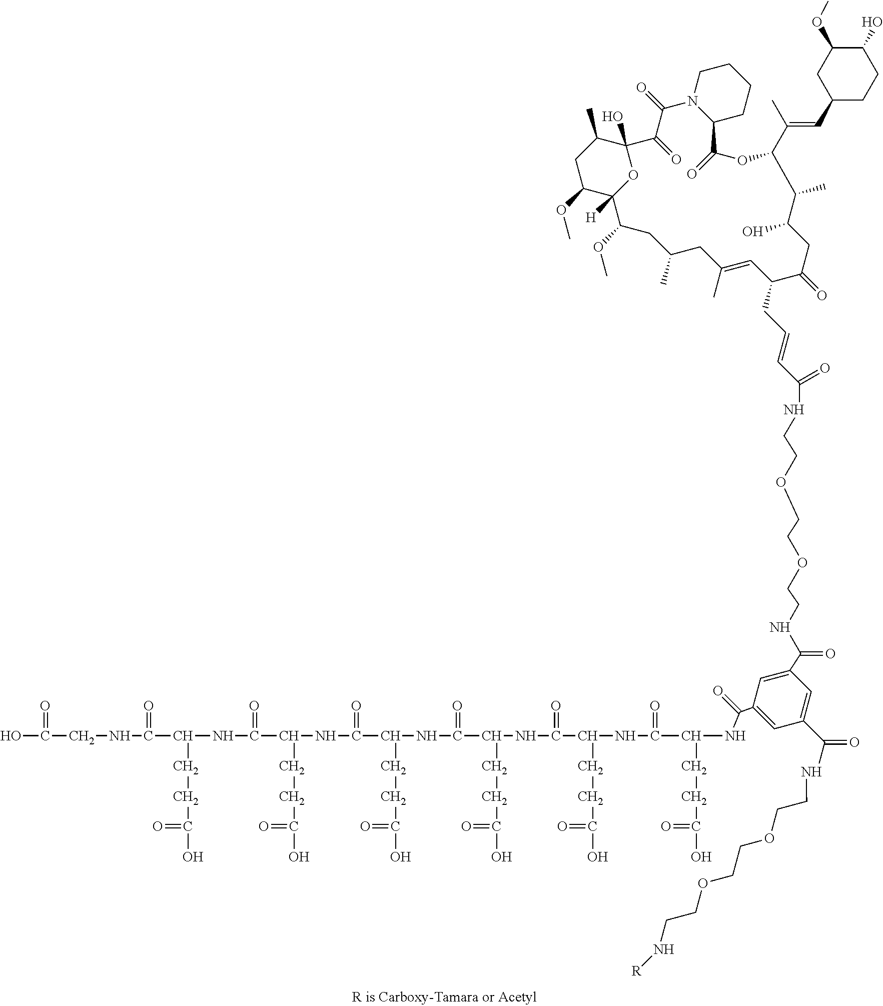

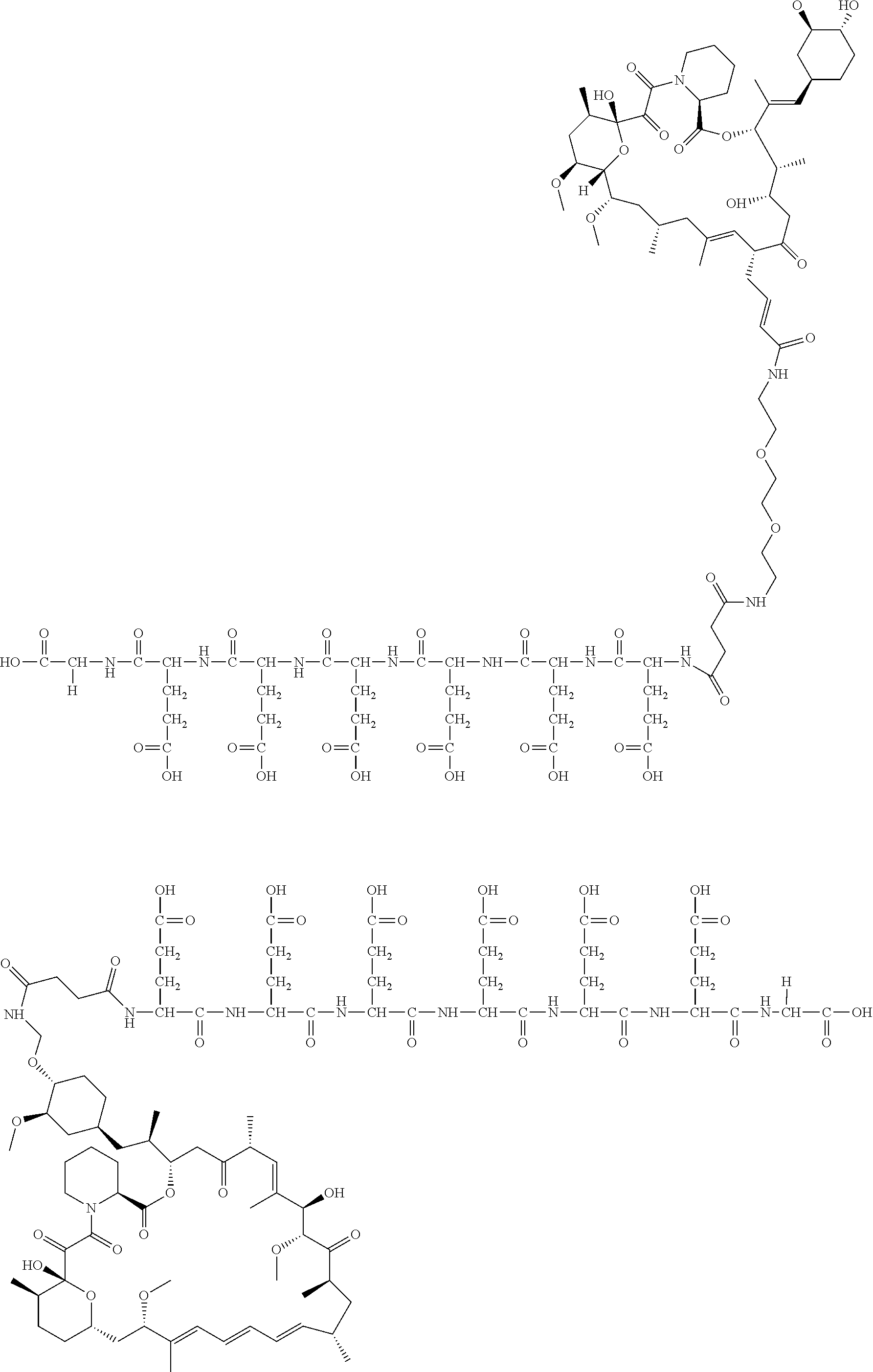

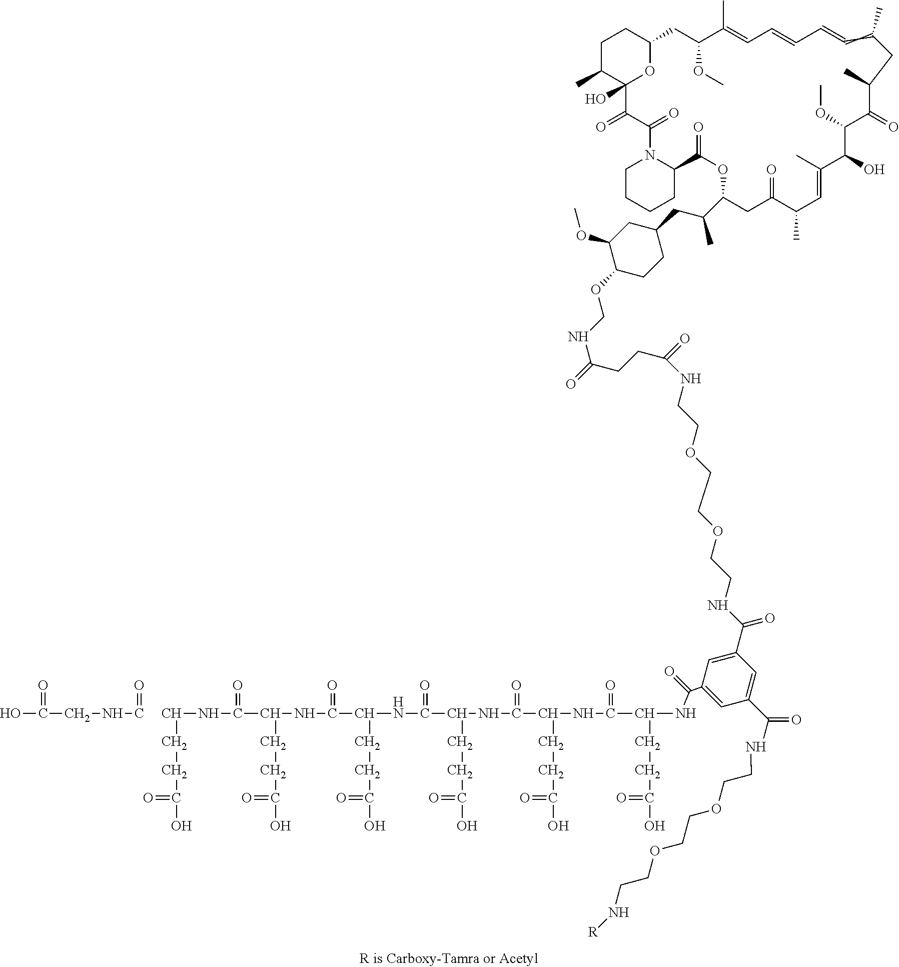

In a further aspect the invention relates to a process for the concentration of active ingredients in an extracellular space of a multicellular object, comprising the steps:
Providing an active ingredient-peptide construct as defined above;
Bringing the construct into contact with a multicellular object.

By an “extracellular space” are meant all the areas which are located outside the cytosol and the membrane surrounding the cytosol. This also includes the culture solution present for example in cell suspensions.

The multicellular object can be any object which consists of at least two identical or different biological cells. The term “biological cell” covers human, animal and also vegetable and bacterial cells as well as monocellular creatures. If the biological cells are bacterial cells or monocellular creatures, then by “multicellular object” is meant a collection of several cells, such as for example a cell colony of a bacterial culture. If the biological cells are human or animal cells, then by “multicellular object” is meant a separated body part, such as for example a transplant, in particular an organ, body part such as a limb or a tissue transplant, blood or a blood fraction, such as for example blood plasma or an in-vitro culture of human and/or animal cells, such as for example a two-dimensional tissue culture or a spheroid culture of the cells. If the biological cells are plant cells, then by “multicellular object” is meant a part of a plant, such as for example leaves, root or stalk or also a whole plant.

In a preferred embodiment the multicellular object is a separated organ or body part, blood or a blood fraction, a cell culture or a plant.

The invention further relates to the use of the active ingredient-peptide construct according to the invention as a medicinal product. The construct according to the invention can be used for the production of medicinal products. The construct according to the invention is preferably used for the treatment of non-immunosuppressive diseases.

The field of application of the medicinal products according to the invention can be the therapy and diagnosis of diseases but also cosmetic in nature, wherein by therapy is meant in the broadest sense also the treatment of pests in the animal and plant kingdom or the support of healing processes in the animal and plant kingdom but also the influencing of biological processes in the desired manner. Particular advantages lie in veterinary and human medicine, in the application of substances on or in cell suspensions, tissue cultures, transplants or the whole mammal.

The present invention also relates to the use of the active ingredient-peptide construct according to the invention as a diagnostic aid.

The invention also relates to the use of the construct according to the invention for the production of a medicinal product for the treatment of non-immunosuppressive diseases.

The medicinal product can be administered in any form known to a person skilled in the art to be suitable for the intended purpose. For example the medicinal product can be used in a form selected from the group consisting of injections, infusions, tablets, creams, sprays, capsules, syrups, emulsions, powders, dry chemicals, suppositories or the like. The medicinal product is particularly preferably used in the form of sprays or tablets.

The present invention also relates in a further aspect to a pharmaceutical composition comprising an active ingredient-peptide construct according to the invention. The composition can be any pharmaceutical composition known to a person skilled in the art to be suitable. In a preferred embodiment the pharmaceutical composition is sprays or tablets.

### EXAMPLES AND FIGURES

The present invention will now be described in more detail with the help of the following figures and examples. The figures and examples have a purely illustrative character and in no way limit the scope of the present invention.

There are shown in:

FIG. 1: The cyclosporin derivative [O-carboxymethyl D-Ser]8 CsA (Cs6)

FIG. 2: The cyclosporin derivative Cs9-TAMRA

FIG. 3: The cyclosporin derivative CsM1

FIG. 4: The cyclosporin derivative CsM2

FIG. 5: The cyclosporin derivative CsM3

FIG. 6: The trifunctional linker (MM-50)

FIG. 7: The trifunctional linker (MM-50) bonded with TAMRA

FIG. 8: The cyclosporin derivative MM-218

FIG. 9: The cyclosporin derivative IK-7-39B

FIG. 10: The cyclosporin derivative CsM4

FIG. 11: The cyclosporin derivative CsM5

FIG. 12: The cyclosporin derivative CsM6, wherein R stands for a carboxy-TAMRA or an acetyl radical.

FIG. 13: The cyclosporin derivative CsM7

FIG. 14: The FK506 derivative FKM1

FIG. 15: The FK506 derivative FKM2

FIG. 16: The FK506 derivative FKM3, wherein R stands for a carboxy-TAMRA or acetyl radical.

FIG. 17: The FK506 derivative FKM4

FIG. 18: The rapamycin derivative RPM1

FIG. 19: The rapamycin derivative RPM2

FIG. 20: The rapamycin derivative RPM3, wherein R stands for a carboxy-TAMRA or acetyl radical.

FIGS. 21A,B: Control images: Hole cells without added cyclosporin derivative taken by means of phase contrast (A) and fluorescence (B).

FIGS. 21C,D: MM218 incubation: Hela cells incubated with 250 nM MM218 for 2 h taken by means of phase contrast (C) and fluorescence (D).

FIGS. 21E,F:Cs9-Rhd incubation: Hela cells incubated with 250 nM Cs9-Rhd for 2 h taken by means of phase contrast (F) and fluorescence (F).

FIG. 22: Influence of MM218 on the number of CD4-positive T-cells which migrated through ovalbumin sensitization into the bronchial lining.

FIG. 23: Influence of MM218 on the number of eosinophilic granulocytes (eosinophiles) which migrated through ovalbumin sensitization into the bronchial lining.

FIG. 24: Chemotaxis assay. It is shown that without any addition of a stimulus (−) a chemotaxis index of roughly 2.7+/−0.3 is obtained.

### EXAMPLE 1

### Cyclosporin Derivatives Syntheses

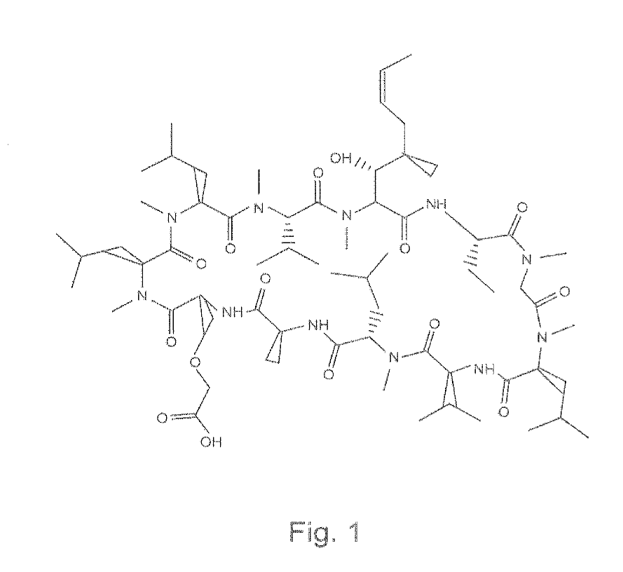

a) [O-carboxymethyl D-Ser]8 CsA (Cs6) (FIG. 1)

A mixture of 60 mg [D-Ser]8 CsA, 20 mg tert-butylbromoacetate and 5 mg benzyltriethyl ammoniumchloride, 1 ml CH₂Cl₂ and 2 ml 30% NaOH was stirred for two hours at room temperature. 10 ml water was then added to the mixture which was then extracted twice with ether. Then the organic solvent layer was dried with Na₂SO₄ and 60 mM KOH in methanol then added without further separation and the mixture stirred for a further three hours at room temperature. After acetic acid was added, the supernatant was removed under vacuum. Ethyl acetate was then added and the mixture washed with water. After separating off the organic layer and drying with Na₂SO₄ followed by vacuum drying, the product was separated off by means of RP HPLC. The mass [M+H]⁺ of the compound was measured at 1276.8 by means of MALDI mass spectrometry.

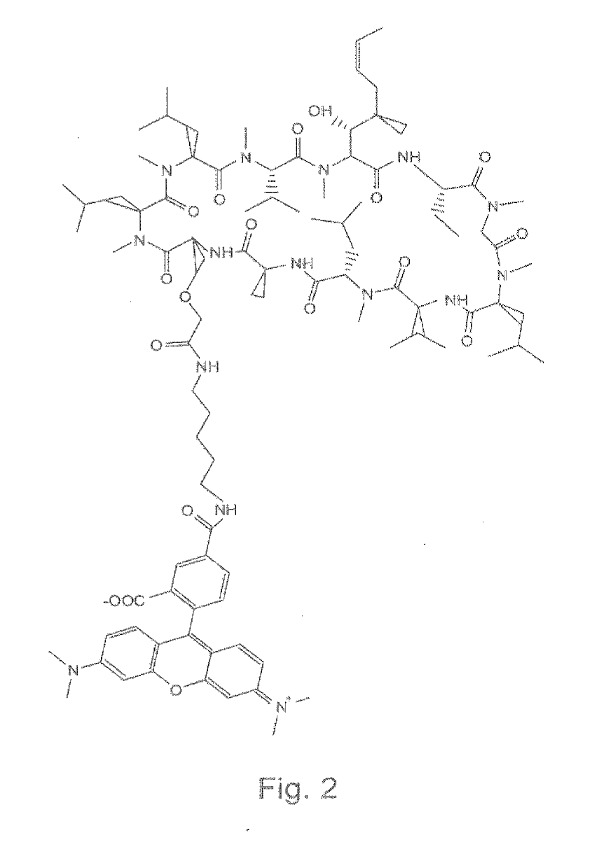

b) Rhodamine-Labelled Cs9-TAMRA (FIG. 2)

100 mg Cs6, 3 parts NH₂(CH₂)₅NHBoc, 4 parts PyBop (benzotriazol-1-yl-oxy-tris-pyrrolidino-phosphonium hexafluorophosphate) and 8 parts DIPEA (N,N-diisopropylethylamine) are stirred overnight in 5 ml CH₂Cl₂ at room temperature. 40 ml ethyl acetate is then added and the organic layer washed with 5% NaHSO₄, 5% NaHCO₃ and saturated NaCl solution. After drying with Na₂SO₄ followed by vacuum drying, the product Cs9 can be separated off by means of HPLC. MALDI mass spectrometry produced a mass [M+H]⁺ of 1461.3 (calculated to: 1460). The substance was then incubated with 5 ml ZnCl₂/ether under a nitrogen blanket for three hours at room temperature. After the addition of 0.1 ml water and 15 ml acetonitrile the precipitated salt could be filtered off.

After vacuum drying and separating-off by means of C8 HPLC a product with the mass [M+H]⁺ of 1361.3 (theoretical: 1360.1) could be obtained by means of MALDI mass spectrometry. A solution, mixed for 15 minutes, consisting of 13.9 mg 5(6)-carboxytetramethylrhodamine (TAMRA) in 2 ml DMF, 12.3 mg HATU ((2-(7-Aza-1H-benzotriazol-1-yl)-1,1,3,3-tetramethyluronium hexafluorophosphate) and 15 μl DIPEA was then added to 40 mg of this substance (Cs9). The hulk solution was then stirred for three hours at room temperature and then filtered. After disruptive accompanying products had been separated off by means of HPLC, the TAMRA-labelled Cs9 could be determined with a mass of 1773.7 (theoretical m/z 1772.1) by means of MALDI mass spectrometry.

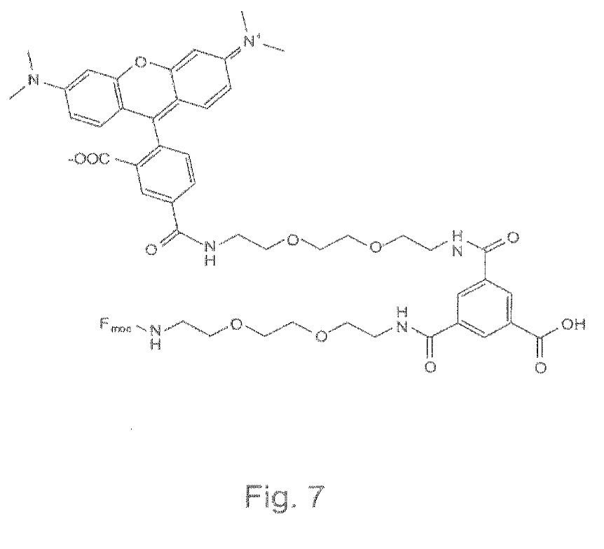

c) TAMRA-Labelled Trifunctional Linker MM-50 (FIG. 7)

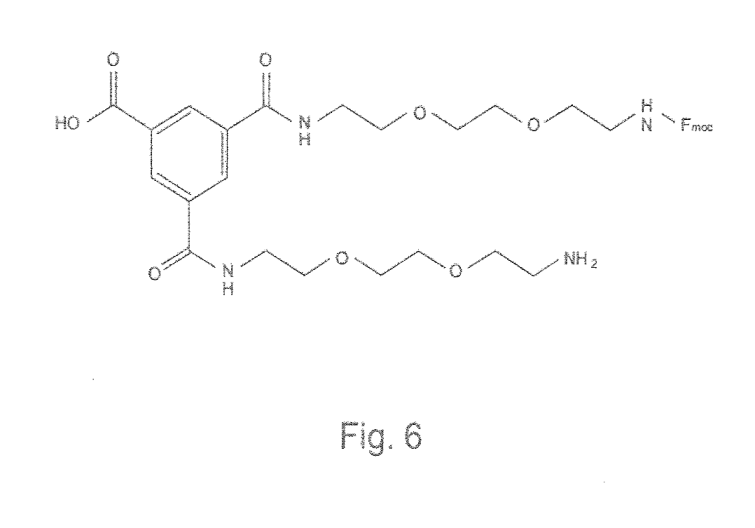

114 mg HATU, 154 μl DIPEA and 82 mg HOAt were added to a solution of 5(6)-carboxytamra (130 mg) in 5 ml DMF. The solution was then mixed for 20 minutes at room temperature and added to a solution of 200 mg of the trifunctional linker MM-50 (FIG. 6; Malesevic M, Lücke C, Jahreis G (2005), Simple and efficient synthesis of new trifunctional templates, Peptides 2004, Proceedings of the Third International and Twenty-Eighth European Peptide Symposium, Kenes International, Israel, 391-392) in 5 ml DMF. After mixing the solution for two hours at room temperature and filtering, the DMF was removed under vacuum. After separating off the product by means of RP HPLC a mass of 1105.2 [M+H]⁺ (theoretical m/z calculated to 1104.5) could be determined by means of MALDI mass spectrometry.

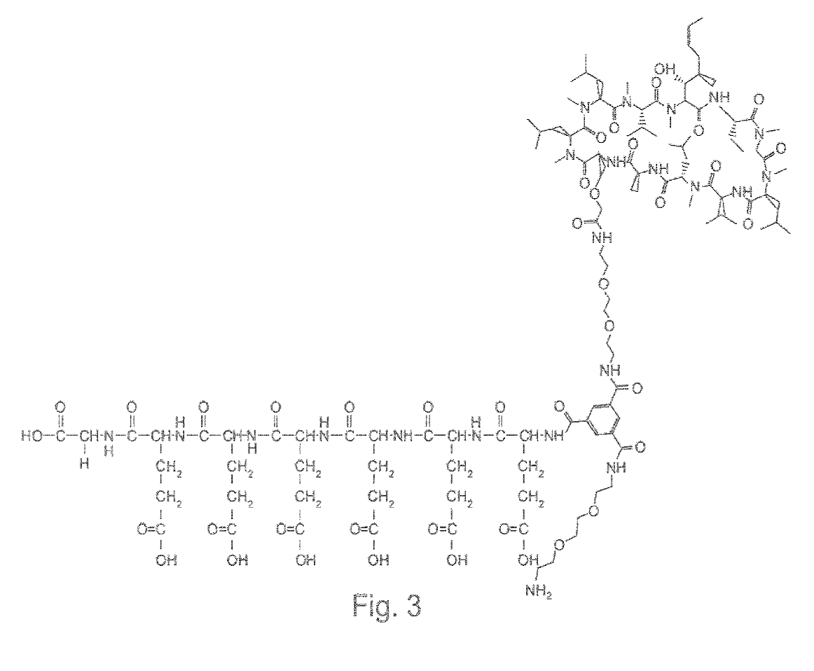

d) Cyclosporin Derivative CsM1 (FIG. 3)

CsM1 was produced by means of standard Fmoc procedures using a 2-ClTrt matrix. In each cycle the Fmoc-protected amino acids are activated with PyBOP and DIPEA in DMF and then coupled for two hours. The Fmoc protective group is split off in each case with 20% piperidine in DMF. The Tamra-labelled trifunctional linker was coupled on overnight as described above. The cyclosporin derivative (Cs6) was pre-activated and coupled overnight with HATU, HOAt and DIPEA. The side chain of the D-glutamic acid was protected as t-butyl ester. After synthesis the product was removed at 5° C. from the matrix by means of 50% TFA/CH₂Cl₂ and isolated by means of RP HPLC.

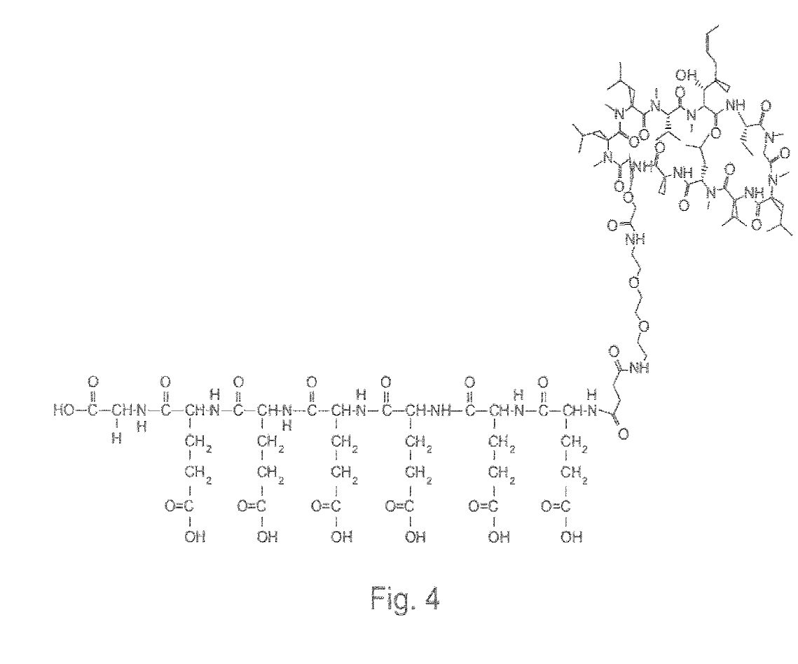

e) Cyclosporin Derivative CsM2 (FIG. 4)

100 mg Cs6, 20 parts NH₂(CH₂₋CH₂—O)₂CH₂CH₂NH₂ and 1.1 parts PyBop were stirred overnight at room temperature in 5 ml DMF. 40 ml ethyl acetate was then added and the organic layer washed with 5% NaHSO₄, 5% NaHCO₃ and saturated NaCl solution. After drying with Na₂SO₄ followed by vacuum drying, the product (CsM2a) was separated off by means of HPLC. CsM2a was then stirred overnight at room temperature with 5 parts succinic anhydride and 10 parts DIPEA in 5 ml DMF. 40 ml ethyl acetate was then added and the organic layer washed with 5% NaHSO₄, 5% NaHCO₃ and saturated NaCl solution. After drying with Na₂SO₄ followed by vacuum drying, the product (CsM2b) was separated off by means of HPLC. CsM2b was then stirred for 10 min at room temperature with 1 part HATU, 3 parts DIPEA in 3 ml DMF. The solution was then added to a mixture of an equivalent part of H(D-Glu)₆-Gly-OH dissolved in 2 ml DMF and stirred overnight. After filtering and preparative HPLC the product CsM2 could be obtained.

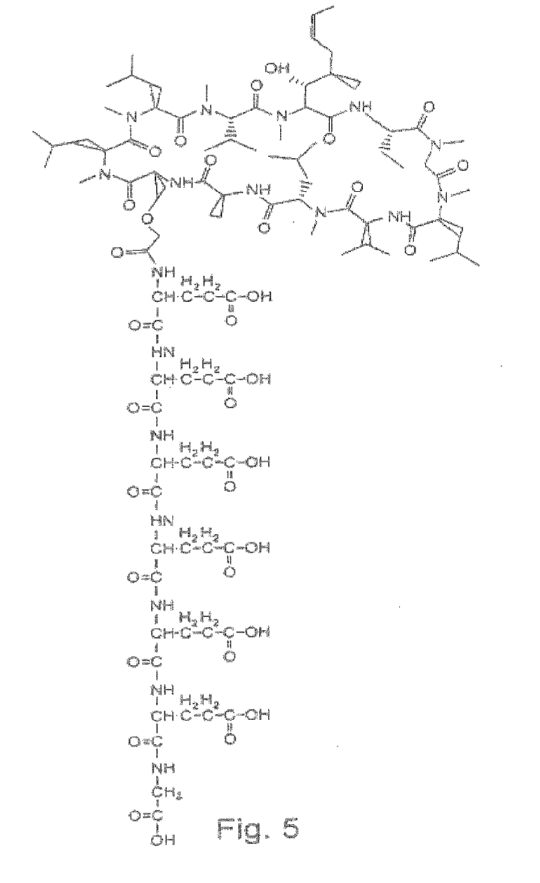

f) Cyclosporin Derivative CsM3 (FIG. 5)

Cs9 was stirred for 20 min at room temperature with 1 part HATU and 3 parts DIPEA in 3 ml DMF. The solution was then added to a mixture of an equivalent part of H(D-Glu)₆-Gly-OH dissolved in 2 ml DMF and stirred overnight. After filtering and preparative HPLC the product CsM3 could be obtained.

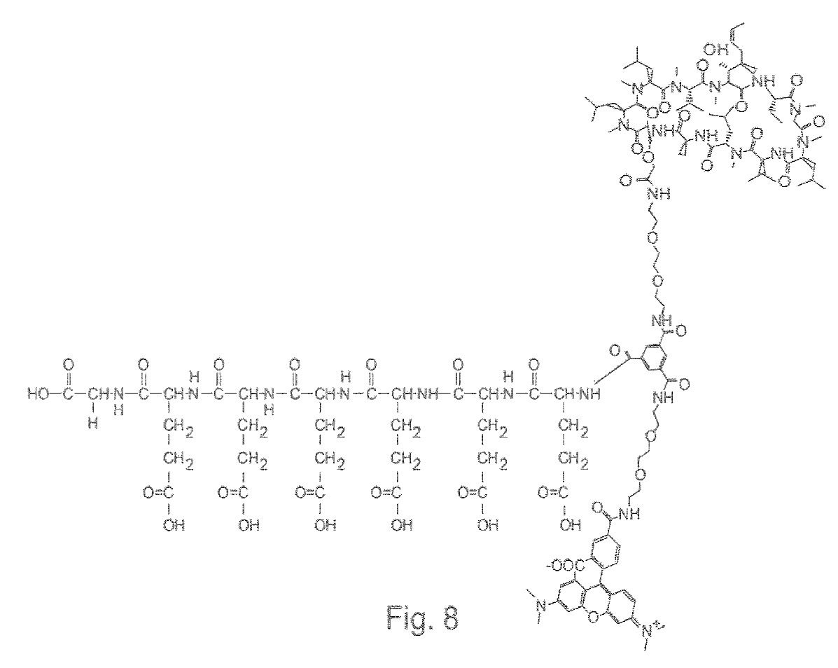

g) Cyclosporin Derivative MM-218 (FIG. 8)

MM-218 was produced by means of standard Fmoc procedures using a 2-ClTrt matrix. In each cycle the Fmoc-protected amino acids are activated with PyBOP and DIPEA in DMF and then coupled for two hours. The Fmoc protective group is split off in each case with 20% piperidine in DMF. The Tamra-labelled trifunctional linker was coupled on overnight as described above. The cyclosporin derivative (Cs6) was pre-activated and coupled overnight with HATU, HOAt and DIPEA. The side chain of the D-glutamic acid was protected as t-butyl ester. After the synthesis the product was removed from the matrix at 5° C. by means of 50% TFA/CH₂Cl₂ and isolated by means of RP HPLC. A mass [M+H]⁺ of 2972.4 (calculated to 2971.5) could be ascertained by means of MALDI mass spectrometry.

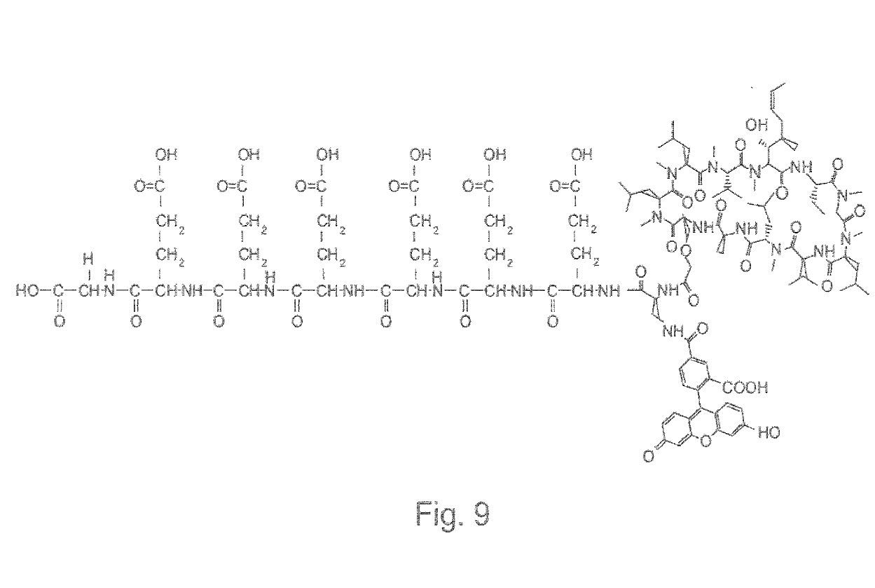

h) Cyclosporin Derivative IK-7-39B (FIG. 9)

H-Dap(fluorescein)-(D-Glu)₆-Gly-OH was synthesized by means of conventional Fmoc chemistry using a 2-ClTrt matrix. With each synthesis cycle, Fmoc-protected amino acids were firstly pre-activated with PyBOP and DIPEA in DMF and then coupled for two hours. The side chain of the D-glutamic acid was protected as t-butyl ester. The Fmoc protective group was split off by means of 20% piperidine in DMF. After the synthesis, the product was split off at room temperature from the matrix with 50% TFA/CH₂Cl₂ and isolated by means of RP HPLC. A mass [M+H]⁺ of 1294.3 (calculated to 1293.4) could be ascertained by means of MALDI mass spectrometry. The H-Dap(fluorescein)-(D-Glu)₆-Gly-OH was then added to a solution of the cyclosporin derivative 6 (Cs6) in DMF, to which 0.9 parts HATU and 3 parts DIPEA were added and mixed for 30 minutes, and stirred overnight. The product IK-7-39B could be separated off by means of RP HPLC. The mass ([M+H]⁺, ascertained with MALDI mass spectrometry, was 2554.0 (calculated to 2552.8).

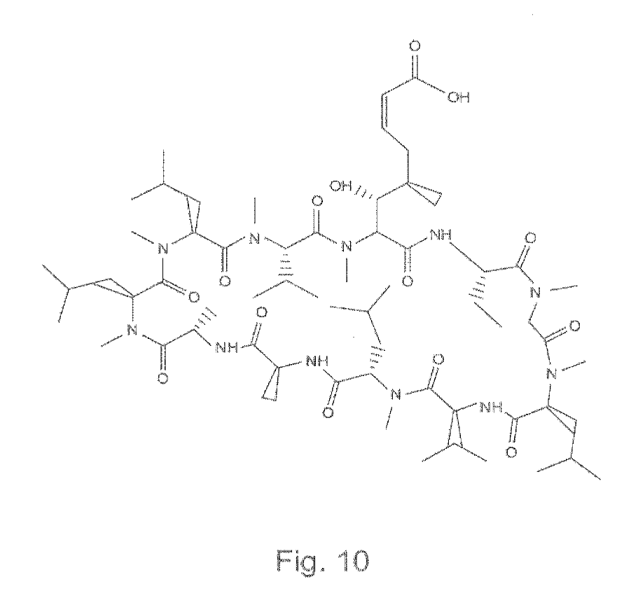

i) Cyclosporin Derivative CsM4 (FIG. 10)

The derivatization of the cyclosporin at position 1 is achieved by boiling cyclosporin A and 0.1 parts “Hoveyda-Grubbs catalyst second generation” (1,3-Bis-(2,4,6-trimethylphenyl)-2-imidazolidinylidene)di-chloro(o-isopropoxyphenylmethylene)ruthenium) and 20 parts dimethyl maleate in toluene accompanied by reflux cooling for 45 h. The toluene is then removed under vacuum and the residue dissolved in DCM/MeOH (10:0.5) and filtered through silica gel. After removing the solvent (under vacuum), 5 ml of a mixture of 2.5 ml 0.2 M LiOH in water and 2.5 ml THF are added and stirred overnight. After neutralization with HCl the product can be isolated by means of preparative HPLC.

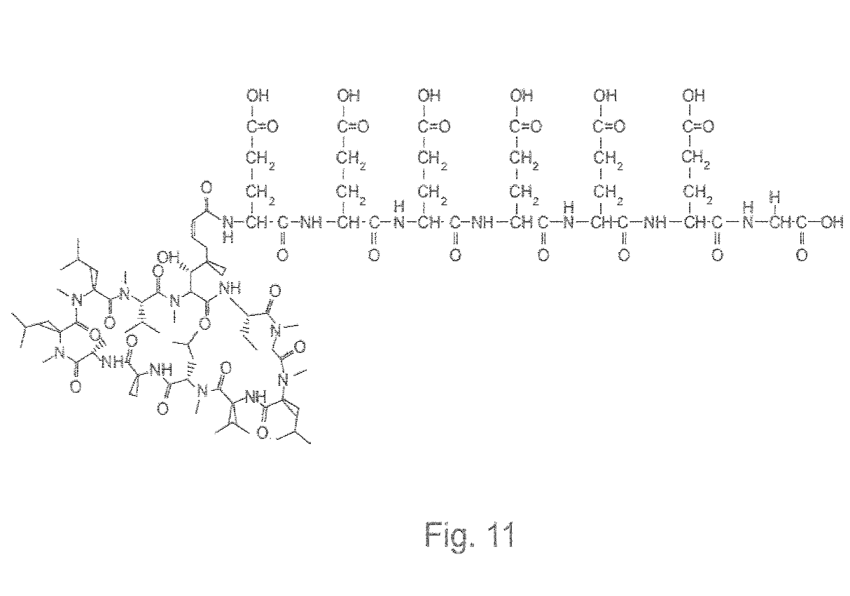

j) Cyclosporin Derivative CsM5 (FIG. 11)

CsM4, 1 part HATU and 3 parts DIPEA in 3 ml DMF are stirred for 20 min at room temperature. The solution is then added to one part H-(D-Glu)₆-Gly-OH dissolved in 2 ml DMF and stirred overnight. After filtration the product can be isolated by means of preparative HPLC.

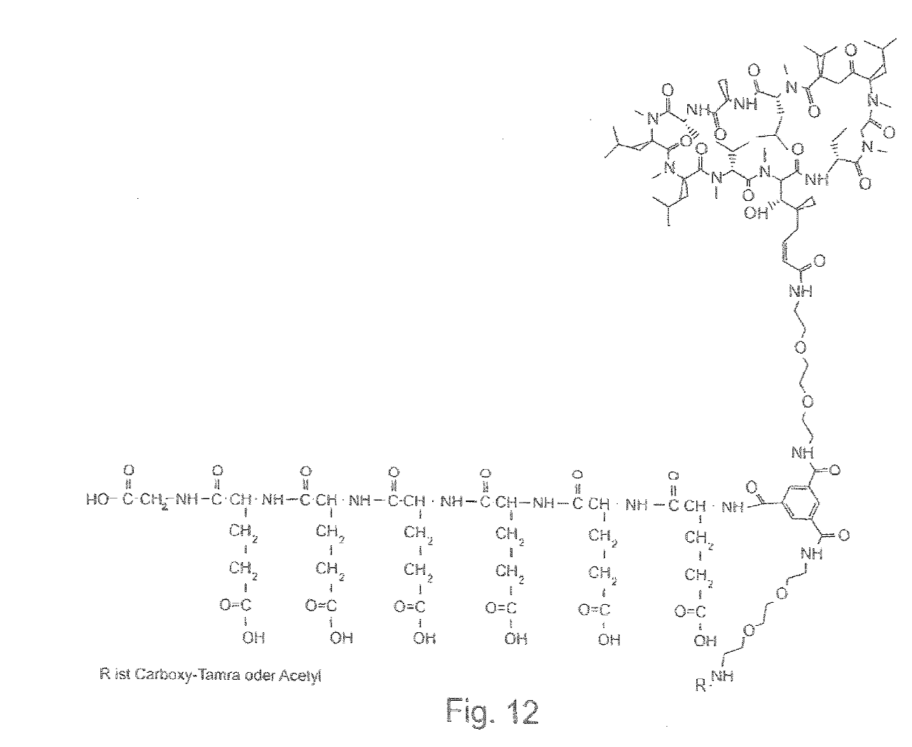

k) Cyclosporin Derivative CsM6 (FIG. 12)

The peptide is synthesized by means of conventional Fmoc chemistry using a 2-ClTrt matrix. With each synthesis cycle, Fmoc-protected amino acids are coupled for two hours with PyBOP and DIPEA in DMF, The Fmoc protective group is split off in each case with 20% piperidine in DMF. The trifunctional linker (Example 1c) is coupled on overnight. The side chain of the D-glutamic acid is protected as t-butyl ester. HATU, HOAt and DIPEA are added to Cs6 (FIG. 1) and the mixture stirred overnight. The product CsM6 can be obtained after splitting-off from the matrix with 50% TFA/CH₂Cl₂ at 5° C. and purification by means of RP HPLC.

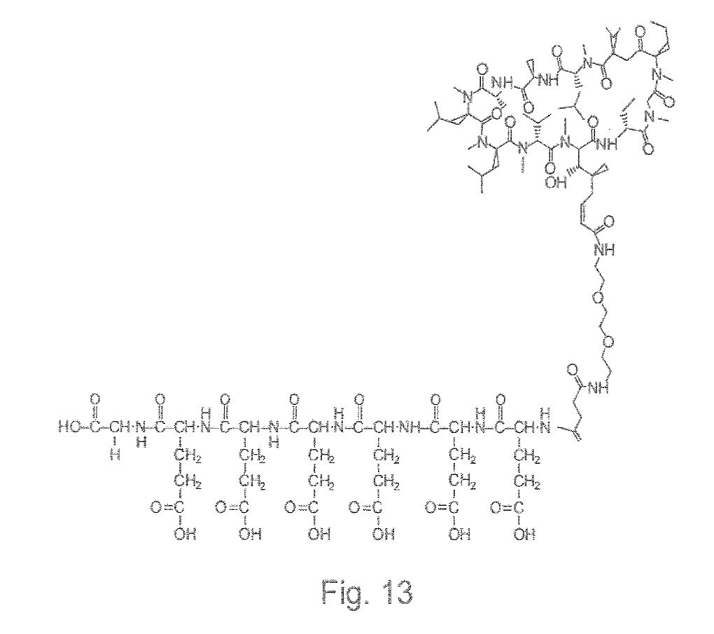

l) Cyclosporin Derivative CsM7 (FIG. 13)

CsM4 (FIG. 10) 20 parts NH (CH₂—CH₂—O)₂CH₂CH₂NH₂ and 1.1 parts PyBop in DMF are stirred overnight at room temperature. 40 ml ethyl acetate is then added and the organic layer washed with 5% NaHSO₄, 5% NaHCO₃ and saturated NaCl solution. After drying with Na₂SO₄ followed by vacuum drying, the product (CsM7a) is separated off by means of HPLC. CsM7a is then stirred at room temperature for 10 min with 1 part HATU, 3 parts DIPEA in 3 ml DMF. The solution is then added to a mixture of an equivalent part of H(D-Glu)₆-Gly-OH dissolved in 2 ml DMF and the mixture stirred overnight. After filtration and preparative HPLC the product CsM7 can be obtained.

### EXAMPLE 2

### FK506 Derivatives

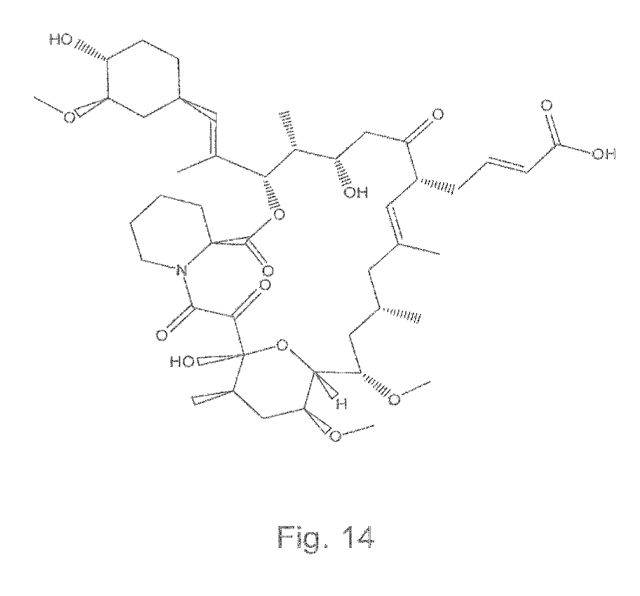

a) FK506 Derivative FKM1 (FIG. 14)

3 mg FK506, 100 μl t-butyl acrylate and 0.5 mg “Grubbs catalyst second generation” (benzylidene[1,3-bis(2,4,6-trimethylphenyl)-2-imidazolidinylidene]dichloro(tricyclohexyl-phosphine)ruthenium) are boiled in CH₂Cl₂ under protective gas (argon) for 5 h accompanied by reflux cooling. The solution is then filtered and dried under vacuum. The residue is then taken up in 2 ml of a solution of 48% TFA, 50% CH₂Cl₂ and 2% TIS (triisopropylsilane) and the mixture stirred for 30 min. After drying under vacuum the product can be isolated by means of RP HPLC.

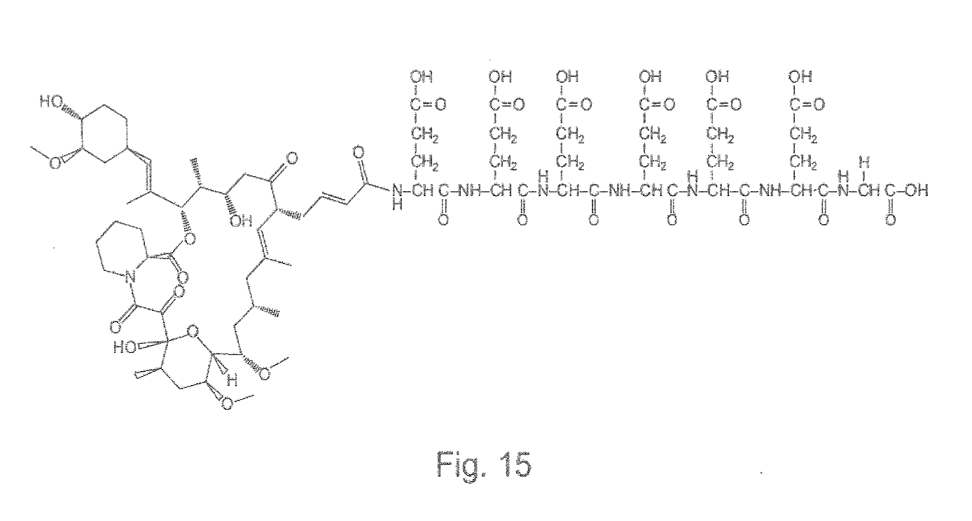

b) FK506 Derivative FKM2 (FIG. 15)

3 ml DMF is added to FKM1 (FIG. 14), 1 part HATU and 3 parts DIPEA and the mixture stirred for 20 min at room temperature. A solution which contains one part H-(D-Glu)₆-Gly-OH in 2 ml DMF is then added and the mixture stirred overnight. After filtering off insoluble constituents the product (FKM2) can be obtained by means of preparative HPLC.

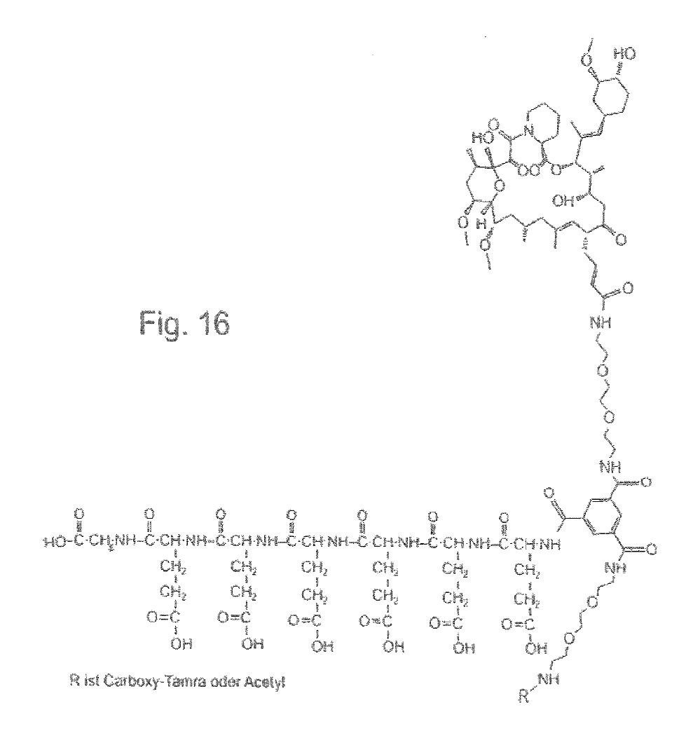

c) FK506 Derivative FKM3 (FIG. 16)

The peptide is synthesized by means of conventional Fmoc chemistry using a 2-ClTrt matrix. With each synthesis cycle, Fmoc-protected amino acids are firstly pre-activated with PyBOP and DIPEA in DMF and then coupled for 2 h. The trifunctional linker (Example 1c) is coupled on overnight. The Fmoc protective group is split off in each case with 20% piperidine in DMF. The side chain of the D-glutamic acid is protected as t-butyl ester. HATU, HOAt and DIPEA are added to FKM1 (FIG. 14) and the mixture stirred overnight. The product

FKM3 can be obtained by means of RP HPLC after splitting-off from the matrix with 50% TFA/CH₂Cl₂ at 5° C. and purification.

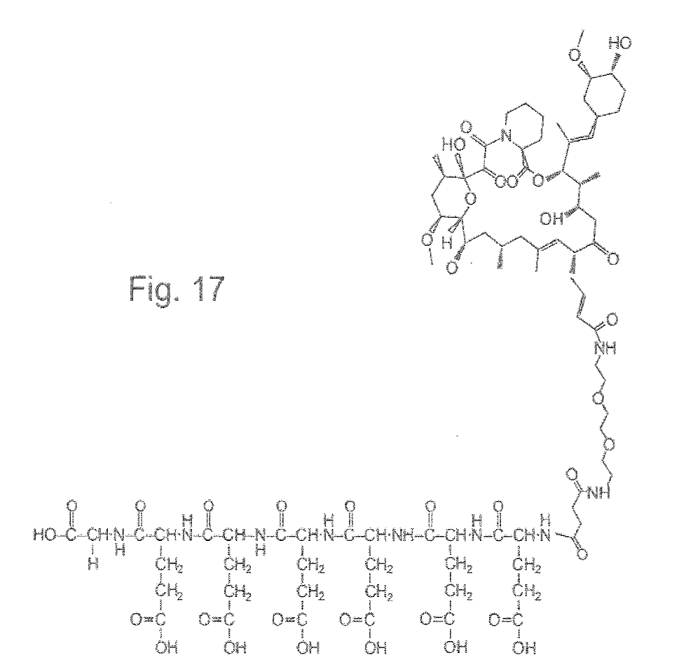

d) FK506 Derivative FKM4 (FIG. 17)

FKM1 (FIG. 14), 20 parts NH₂(CH₂—CH₂—O)₂CH₂CH₂NH₂ and 1.1 parts PyBop in DMF are stirred overnight at room temperature. 40 ml ethyl acetate is then added and the organic layer washed with 5% NaHSO₄, 5% NaHCO₃ and saturated NaCl solution. After drying with Na₂SO₄ followed by vacuum drying, the product (FKM4a) is separated off by means of HPLC. FKM4a is then stirred overnight at room temperature with 5 parts succinic anhydride and 10 parts DIPEA in 5 ml DMF.

40 ml ethyl acetate is then added and the organic layer washed with 5% NaHSO₄, 5% NaHCO₃ and saturated NaCl solution. After drying with Na₂SO₄ followed by vacuum drying, the product (FKM4b) is separated off by means of HPLC. FKM4b is then stirred at room temperature for 20 min with 1 part HATU, 3 parts DIPEA in 3 ml DMF. The solution is then added to a mixture of an equivalent part of H(D-Glu)₆-Gly-OH dissolved in 2 ml DMF and the mixture stirred overnight. The product FKM4 can be obtained after filtration and preparative HPLC.

### EXAMPLE 3

### Rapamycin Derivatives

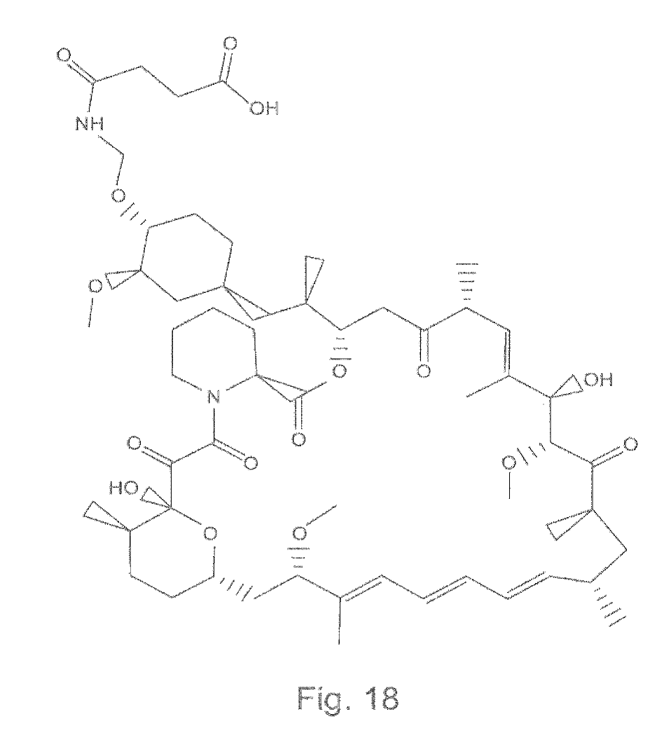

a) Rapamycin Derivative RPM1 (FIG. 18)

A solution of one part rapamycin, 5 parts 2,6 lutidine and 5 parts bromoethyl triflate are incubated in toluene at 65° C. for 18 h. After cooling, saturated sodium bicarbonate solution is added and the product extracted with ethyl acetate. The extraction is repeated three times. The combined extracts are filtered and dried under vacuum. The product (RPM1a) is then isolated by means of preparative HPLC and taken up in DMF. After 1.2 parts sodium azide are added the mixture is stirred for two hours. After saturated sodium bicarbonate solution is added the solution is extracted three times with acetyl acetate. The combined extracts are then dried over Na₂SO₄, filtered and dried by means of a vacuum. The product (RPM1b) can then be obtained by means of preparative HPLC. RPM1b is then taken up in 70% THE and stirred overnight after the addition of five parts triphenylphospine. After ethyl acetate is added the solution is washed three times with saturated common salt solution and then dried over Na₂SO₄ and filtered. The product (RPM1c) is then isolated by means of preparative HPLC. After dissolution of the RPMic in DMF 1.1 parts succinic anhydride are added and the ph of the solution set to approximately pH 7.5 with diisopropylethylamine. After stirring the mixture overnight, the product RPM1 can be obtained by means of preparative HPLC.

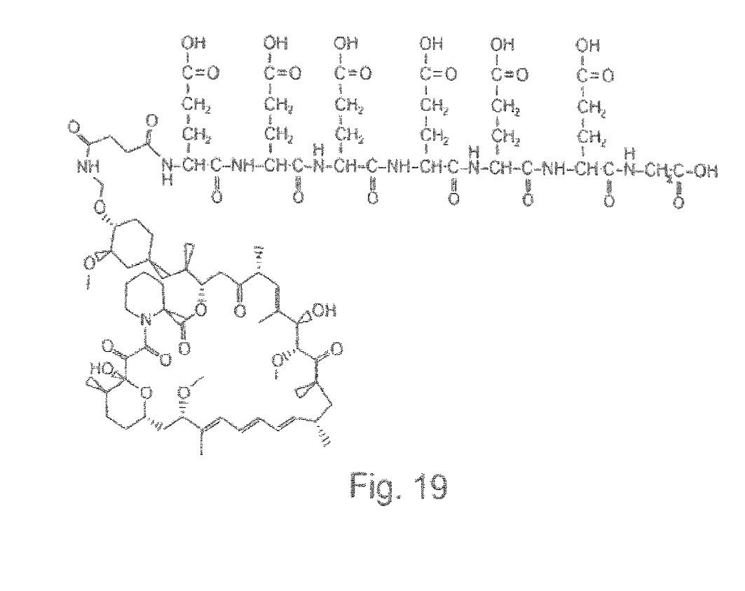

b) Rapamycin Derivative RPM2 (FIG. 19)

3 ml DMF is added to RPM1, one part HATU and 3 parts DIPEA, and the mixture stirred for 20 min at room temperature. One part H-(D-Glu)₆-Gly-OH in 2 ml DMF is then added and the mixture stirred overnight. After filtering off the undissolved residues, the product RPM2 can be isolated by means of preparative HPLC.

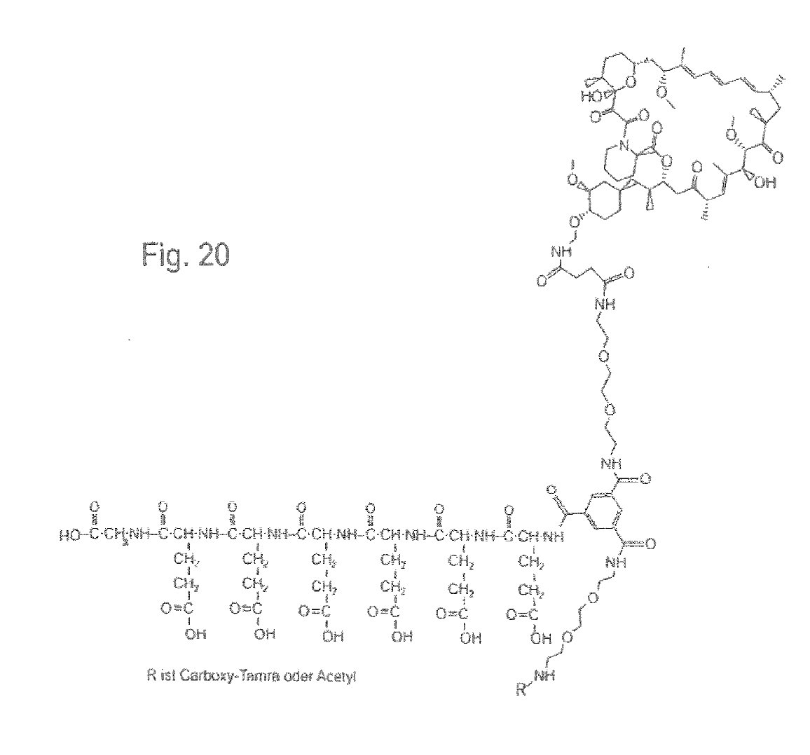

c) Rapamycin Derivative RPM2 (FIG. 20)

The compound is synthesized by means of conventional Fmoc chemistry using a 2-ClTrt matrix. In each cycle the Fmoc-protected amino acids are activated with PyBOP and DIPEA in DMF with a coupling time of two hours. The Fmoc protective group is split off in each case with 20% piperidine in DMF, The trifunctional linker is coupled on overnight. The rapamycin derivative RPM1 is coupled overnight by means of HATU, HOAt and DIPEA. The D-glutamic acid side chain is protected as t-butyl ester. The peptide is then split off from the matrix at 5° C. by means of 50% TFA/CH₂Cl₂ and isolated by means of RP HPLC.

### EXAMPLE 4

### Uptake of Chemically-Modified CsA Compounds by Hela Cells

The advantage of the present invention becomes clear upon investigating the behaviour during transport of two cyclosporin derivatives. The Cs9 derivative MM218 differs from the Cs9 derivative Cs9-Rhd by virtue of the acid peptide added by synthesis. The uptake of chemically-modified, fluorescent

CsA derivatives into living eukaryotic cells is shown on a cell line by means of confocal laser scan microscopy. For the experiment, 10⁵ Hela cells were placed in Ibidi® Petri dishes (μ-Dish, 35 mm, high) and incubated for 1-2 days in DME medium (high glucose) at 37° C. and 5% CO₂.

Examination was by means of an inverted microscope (Nikon ECLIPSE ClTE2000-E) which was equipped with a focussing aid, T-PFS, in order to prevent a so-called focus drift. An objective with phase contrast (40.0× Plan Fluoroil immersion NA 1.30) was used for the pictures, as well as the software associated with the microscope EZ-C1 3.7. The fluorophor 5-(6)-carboxytetramethylrhodamine was excited by a Melles & Griot 561 nm laser.

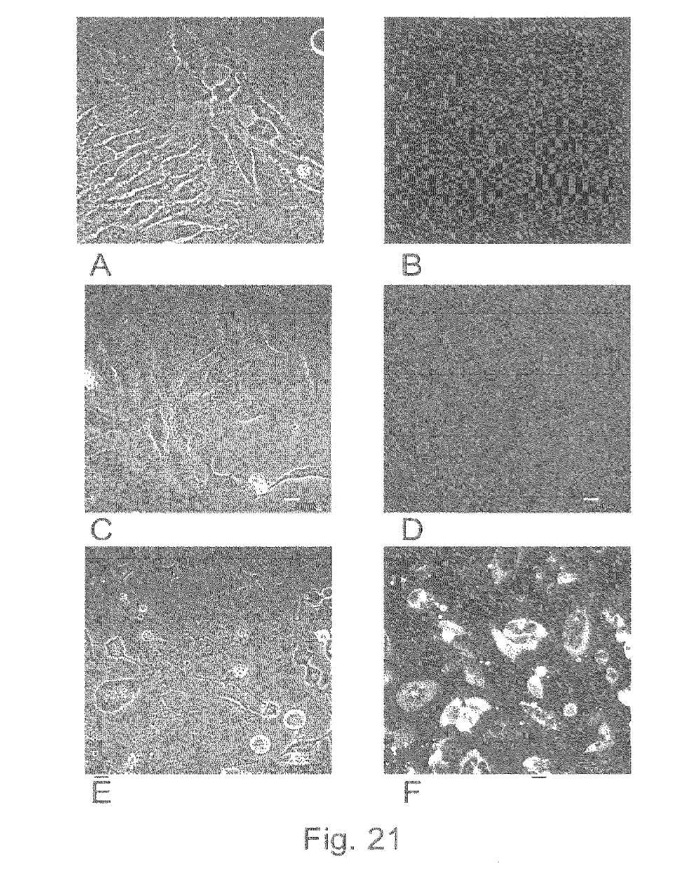

The cells were firstly washed twice with 2 ml PBS pH 7.4 (Dulbecco) and then taken up in 2 ml MIK medium (phenol red-free DME medium, FCS-free, with 20 mM HEPES pH 7.2 and 0.01% carbenicillin) and incubated for 20 min at 37° C. and 5% CO₂ During the taking of the pictures the cells were in an incubator for microscopes (Stage Top Incubator INU series from Tokai Hit®) at 37° C. and 5% CO₂. The investigation was started by adding 250 nM Cs9-Rhd (final concentration) or 250 nM MM218 (final concentration) dissolved in DMSO and diluted in MIK medium. FIG. 20 shows Hela cells incubated with CsA derivatives after 2 h. FIGS. 21A,B: Control images: Hela cells without added cyclosporin derivative by means of phase contrast (A) and fluorescence (B). No structures whatever are visible in the fluorescence light. FIGS. 21C,D: MM218 incubation: Hela cells incubated with 250 nM MM218 for 2 h, by means of phase contrast (C) and fluorescence (D). In the fluorescence light the Hela cells are visible only as shadows in the area around the fluorescent cyclosporin derivative. The cyclosporin derivative provided with an acid peptide is not transported into the Hela cells. FIGS. 21E,F: Cs9-Rhd incubation: Hale cells incubated with 250 nM Cs9-Rhd for 2 h, by means of phase contrast (E) and fluorescence (F). In the fluorescence light the Hela cells are visible as fluorescent cells. The cyclosporin derivative not provided with an acid peptide accumulates within the Hela cells.

### EXAMPLE 5

a) Preparation and Production of Mononuclear Mice Cells

Ectomized mice spleen (BALB/c line) was crushed between two object glasses in order to produce suitable cell suspensions. The thus-obtained suspension was then filtered through a nylon sieve in order to separate off coarse constituents. The thus-obtained cells were centrifuged together with a lymphocyte-separation medium (Mediatech) in order to obtain mononuclear cells. Cell cultures of these cells were then incubated in microtiter plates (8×12 cavities) at a cell density of 6×10⁵ cells per cavity in EHAA medium/5% FCS (Click's medium) in the presence of 10 μg/ml concanavalin A (ConA) with 2 μM MM218 or unmodified cyclosporin A (sigma) or 1% ethanol (dilution/diluent). After a culture time of 48 h 1 μCi 3H-thymidine was added per cavity and incubation continued for a further 6 h. The cells of every cavity were then harvested (TomTec 96-well harvester) and the radioactivity incorporated into the cells measured (Tri-Lux beta-plate counter).

b) Asthma Studies

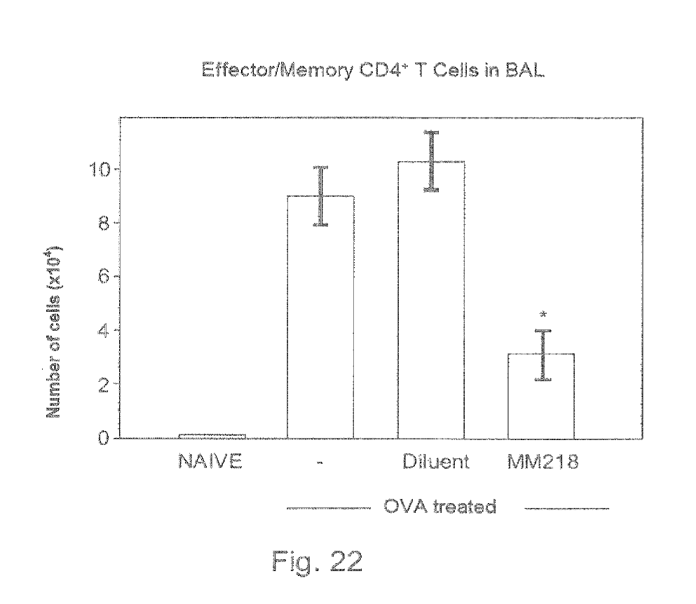

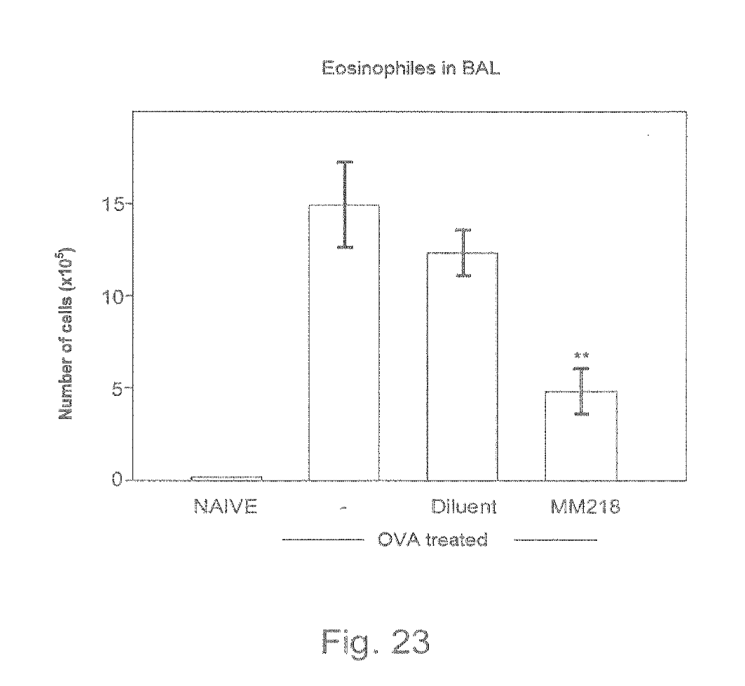

An immune response to ovalbumin is provoked by administering ovalbumin together with aluminium hydroxide. The immune response can be tracked with the help of the T helper cells population (CD4+) which has migrated into the bronchial lining and the eosinophilic granulocytes which have migrated in. Female mice (BALB/c line) were sensitized by intraperitoneal (i.p.) administration of 50 μg ovalbumin (OVA) dissolved in phosphate buffer (PBS) plus 100 μL aluminium hydroxide (alum) with a total volume of 200 μL per mouse on day 0. 100 μg OVA in PBS (50 μL total volume) in each case was then administered intranasally to the OVA/alum-sensitized mice under mild anaesthesia (isoflurane) on days 7-10. From these animals groups were formed which additionally received on days 7, 9, and 11 either 200 μg MM218 in PBS (i.p.), PBS only (diluent) or no further addition (−). On day 12 all the animals were killed by CO₂ exposure and cells of the bronchial tract obtained by bronchial lavage (BAL) by means of a cannula introduced into the trachea washing three times each with 1 ml cold PBS. The obtained cells of the BAL were then double dyed (a) with Cy-Chrome-conjugated anti-mouse CD4-antibodies and (b) with FITC-conjugated anti-mouse CD62L-antibodies. The cells were then analyzed by means of FACS. Effector/Memory CD4⁺T cells were distinguished as CD4⁺/CD62L⁻ lymphocytes and eosinophilic cells with the help of their scattered light properties (FSC/SSC). The results are summarized in FIGS. 22 and 23: FIG. 22: Influence of MM218 on the number of CD4-positive T-cells which migrated through ovalbumin sensitization into the bronchial lining. The untreated mice (naive) served as a control as did the animals sensitized with OVA (−) and those treated only with the MM218 solvent. The administration of MM218 very significantly reduced the number of CD4-positive T-cells. FIG. 23: Influence of MM218 on the number of eosinophilic granulocytes (eosinophiles) which migrated through ovalbumin sensitization into the bronchial lining. The untreated mice (naive) served as a control as did the animals sensitized with OVA (−) and those treated only with the MM218 solvent. The administration of MM218 very significantly reduced the number of eosinophiles.

c) Chemotaxis Assay

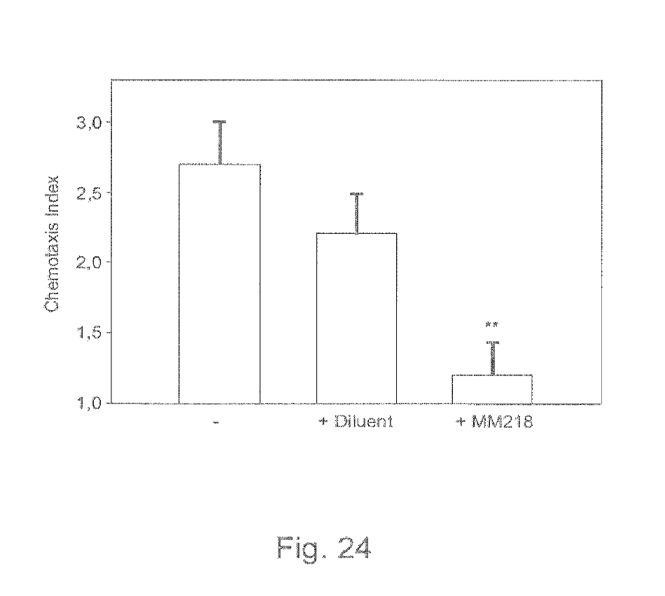

Activated CD4⁺T cells were obtained by stimulating the mononuclear cells (3×10⁶ cells/titer-plate cavity) generated in the proliferation assay with ConA (10 μg/ml) overnight. The CD4⁺ T cells were then purified by means of MACS negative selection kit (Miltenyi Biotec, Auburn Calif.). The chemotaxis assay was carried out in Boyden chambers (Neuroprobe) with 48 cavities, wherein each cavity consists of two compartments, separated by a 5-μm polycarbonate membrane (Neuroprobe). The assays were started by adding 10⁴ cells to the medium (RPMI 1640—1% BSA) of the upper compartment. The medium of the lower compartment contained either 100 ng/ml human cyclophilin A (Calbiochem) or no additives whatever. The influence of active ingredients on cell migration was compared after the addition of these compounds to both compartments. The active-ingredient concentration used was either 2 μM MM218 dissolved in ethanol or 1% ethanol (diluent). The thus-loaded chemotaxis chambers were then incubated at 37° C. in 5% CO² for 50 min. The dividing membranes were then removed and cells which had not migrated were scraped off. The membrane was then dyed with Wright-Giemsa solution (CAMCO, Fort Lauderdale, Fla.). The chemotaxis index was then obtained for each membrane by dividing the number of migrated cells by the number of cells which migrate without any stimulus whatever. FIG. 24 shows that without any addition of a stimulus (−) a chemotaxis index of roughly 2.7+/−0.3 is obtained. The addition of the MM218 solvent (+diluent) shows an insignificant, and the addition of the active ingredient a highly significant, influence on the chemotaxis index.

1. Active ingredient-peptide construct comprising an active ingredient A and a peptide B, wherein the construct has a net charge that is negative at pH 6 and wherein the active ingredient-peptide construct is free from a constituent C which can pass through the membrane of a biological cell.

2. Active ingredient-peptide construct according to claim 1, characterized in that the peptide B is constructed from 2 to 25 amino acids.

3. Active ingredient-peptide construct according to claim characterized in that the peptide B is not proteolytically degradable extracellularly.

4. Active ingredient-peptide construct according to claim 1, characterized in that the amino acids of the peptide B are selected from the group consisting of aspartic acid and glutamic acid.

5. Active ingredient-peptide construct according to claim 1, characterized in that the active ingredient A is a pharmaceutical active ingredient.

6. Active ingredient-peptide construct according to claim 5, characterized in that the active ingredient A is selected from the group consisting of cyclosporin A, FK506 and rapamycin.

7. Active ingredient-peptide construct according to claim 1. characterized in that the active ingredient A is cyclosporin A.

8. Active ingredient-peptide construct according to claim 1, characterized in that the active ingredient A is FK506.

9. Active ingredient-peptide construct according to claim 1, characterized in that the active ingredient A is rapamycin.

10. Active ingredient-peptide construct according to claim 1, characterized in that the peptide B is covalently bonded to the active ingredient A.

11. Active ingredient-peptide construct according to claim 1, characterized in that the construct also comprises an indicator.

12. Active ingredient-peptide construct according to claim 11, characterized in that the indicator is covalently bonded to the active ingredient A.

13. Active ingredient-peptide construct according to claim 11, characterized in that the indicator is covalently bonded to the peptide B.

14. Active ingredient-peptide construct according to claim 11, characterized in that the indicator is covalently bonded to a linker which joins the peptide B to the active ingredient A.

15. Active ingredient-peptide construct according to claim 11, characterized in that the indicator is selected from the group consisting of dyes, voltage-sensitive indicators, pH indicators, calcium-sensitive indicators, radioactive elements, NMR labels, electron-spin labels or combinations thereof.

16. Active ingredient-peptide construct according to claim 1, characterized in that the active ingredient-peptide construct is free from a protease interface.

17. Active ingredient-peptide construct according to claim 1, characterized in that the construct corresponds to the Formula

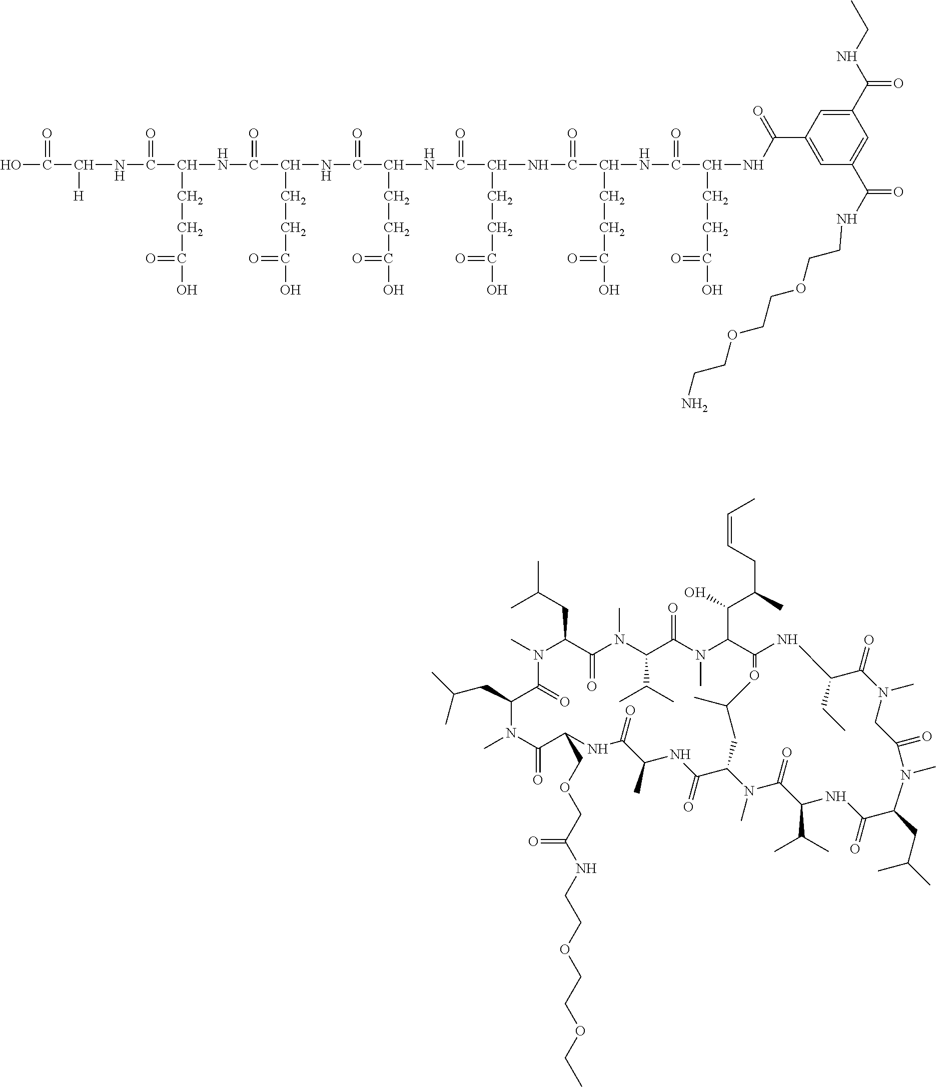

18. Active ingredient-peptide construct according to claim 1, characterized in that the construct corresponds to the Formula

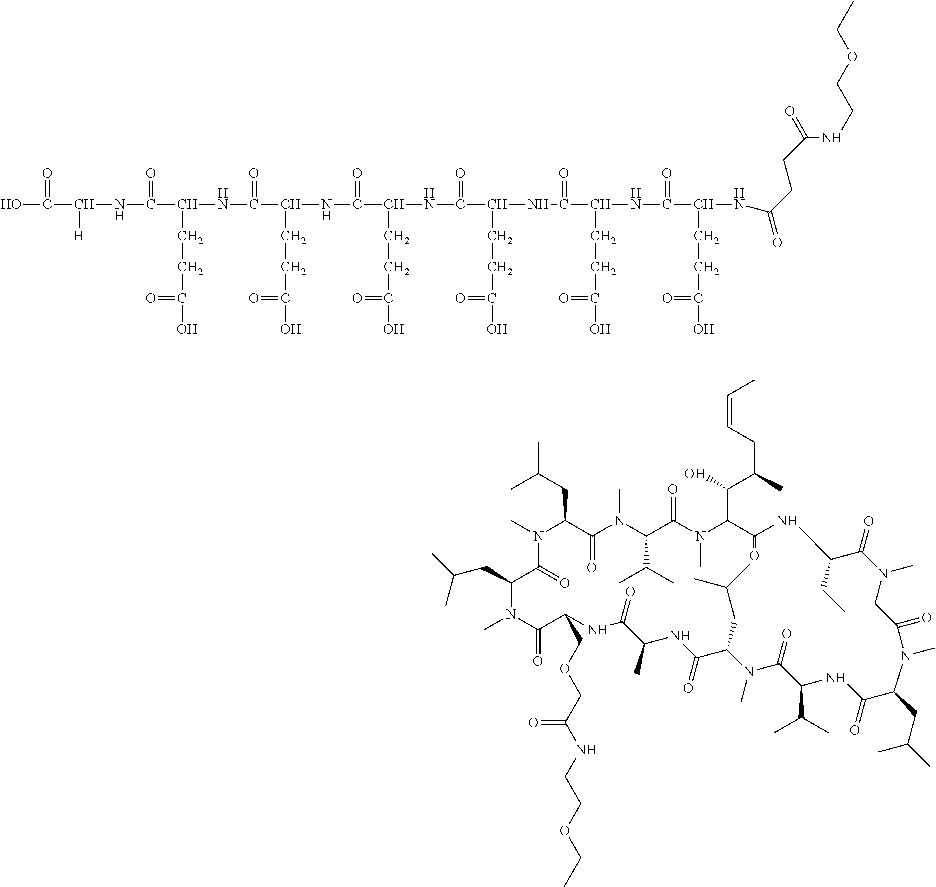

19. Active ingredient-peptide construct according to claim 1, characterized in that the construct corresponds to the Formula

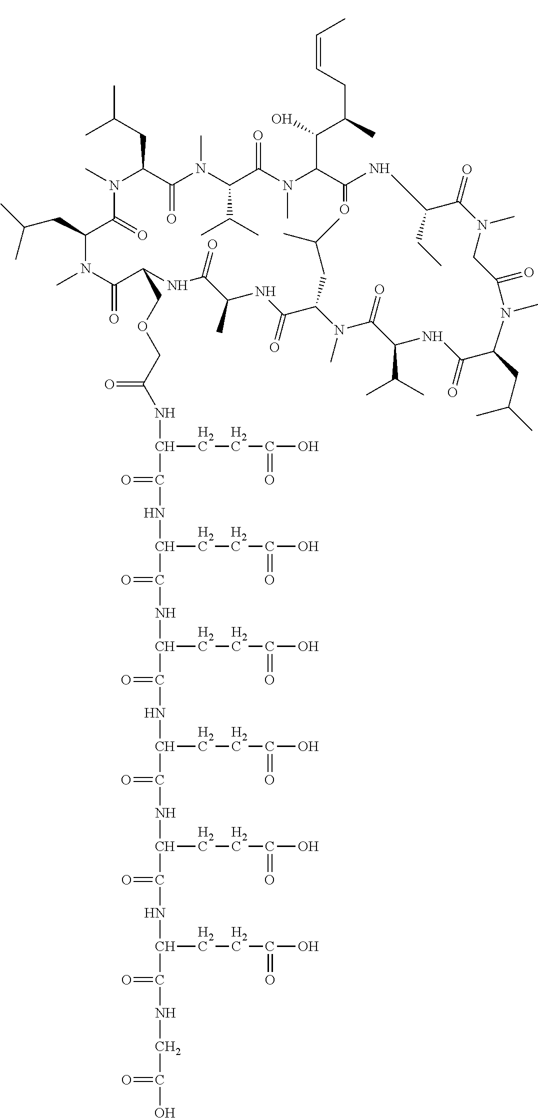

20. Active ingredient-peptide construct according to claim 1, characterized in that the construct corresponds to the Formula

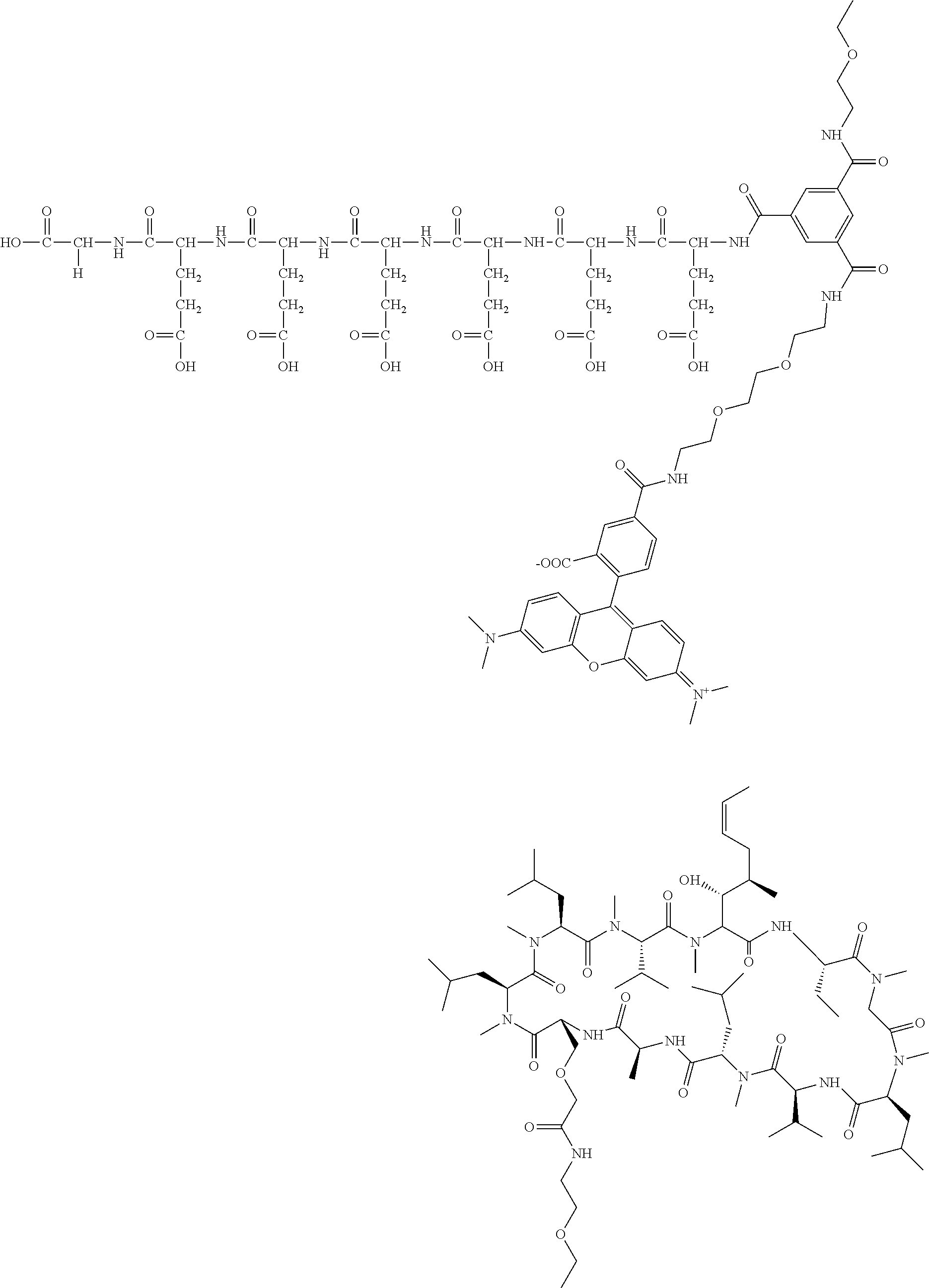

21. Active ingredient-peptide construct according to claim 1, characterized in that the construct corresponds to the Formula

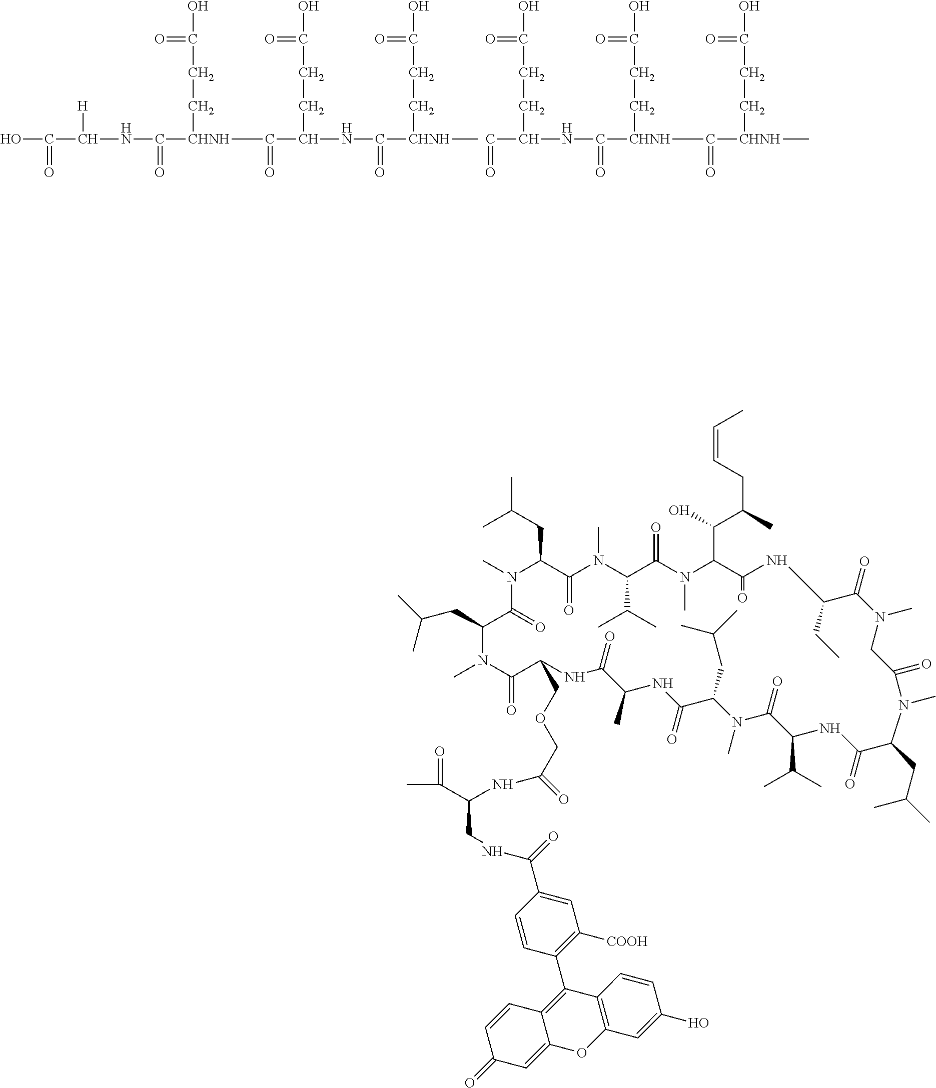

22. Active ingredient-peptide construct according to claim 1, characterized in that the construct corresponds to the Formula

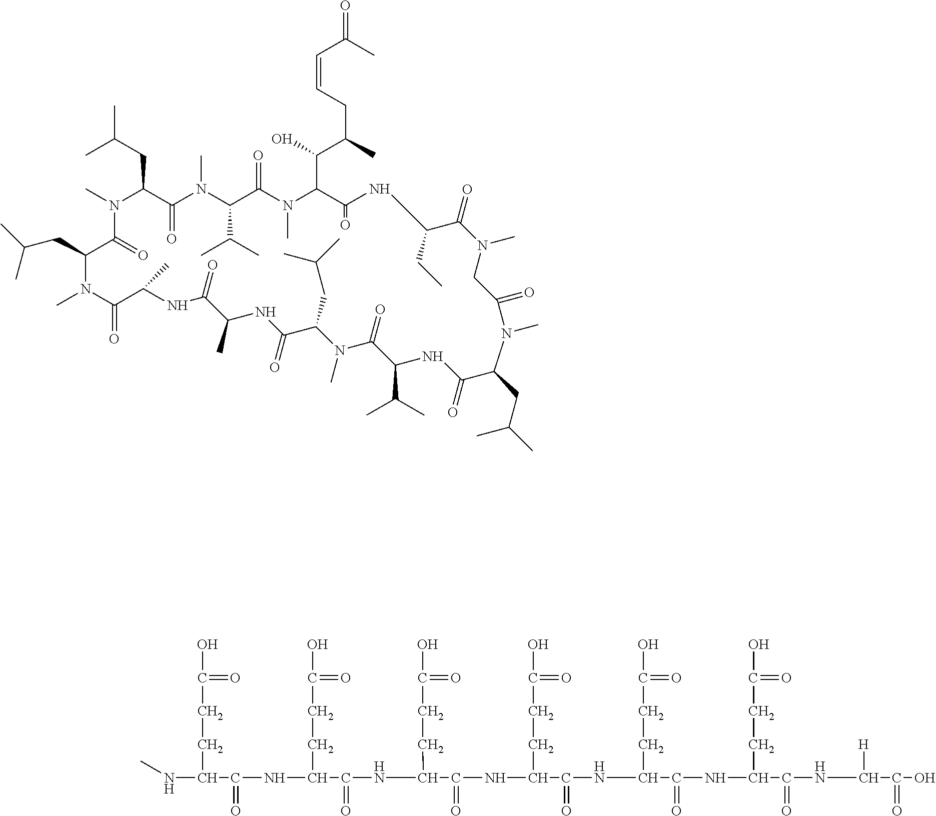

23. Active ingredient-peptide construct according to claim 1, characterized in that the construct corresponds to the Formula 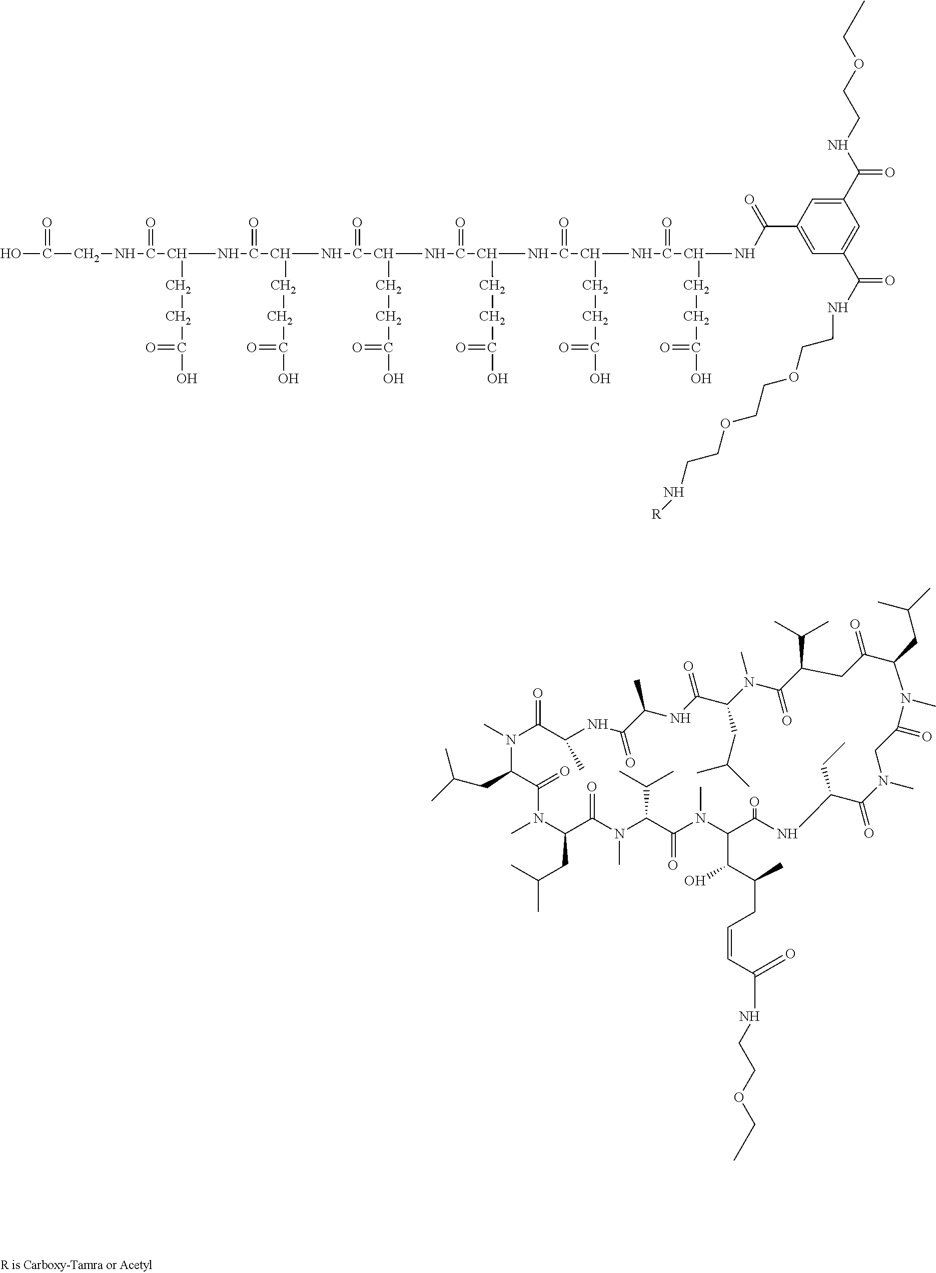
wherein R is a carboxy-tamra or acetyl radical.

24. Active ingredient-peptide construct according to claim 1, characterized in that the construct corresponds to the Formula

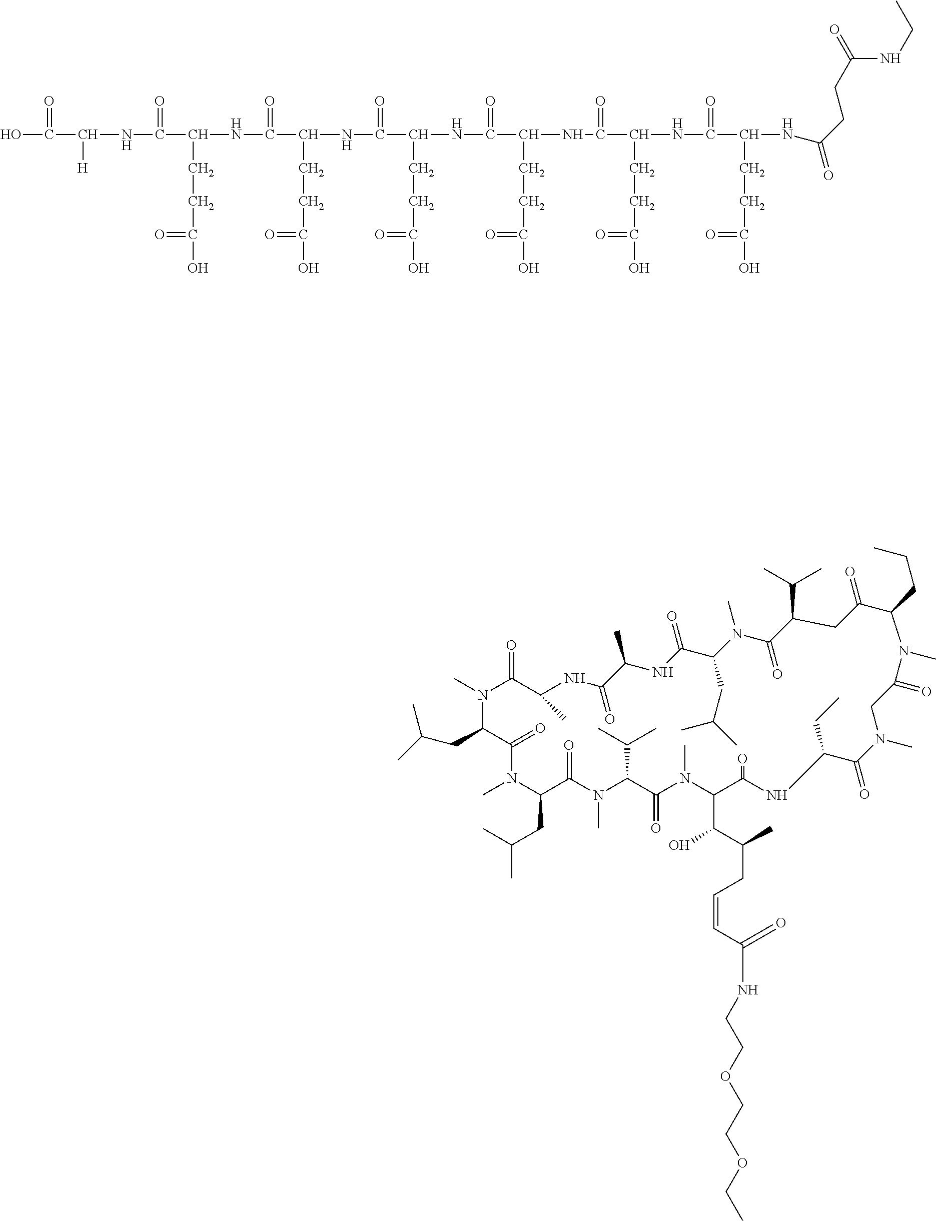

25. Active ingredient-peptide construct according to claim 1, characterized in that the construct corresponds to the Formula

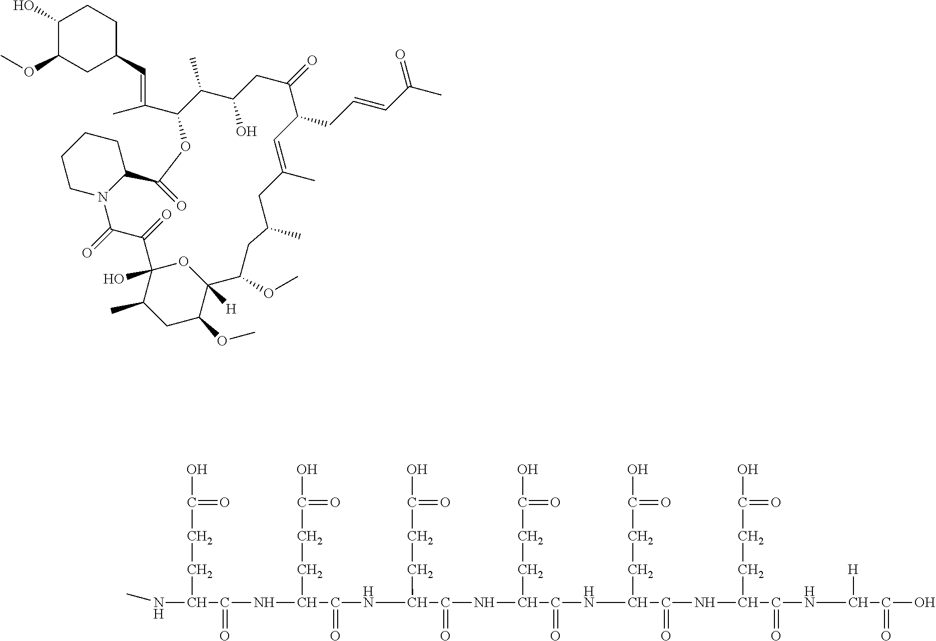

26. Active ingredient-peptide construct according to claim 1, characterized in that the construct corresponds to the Formula 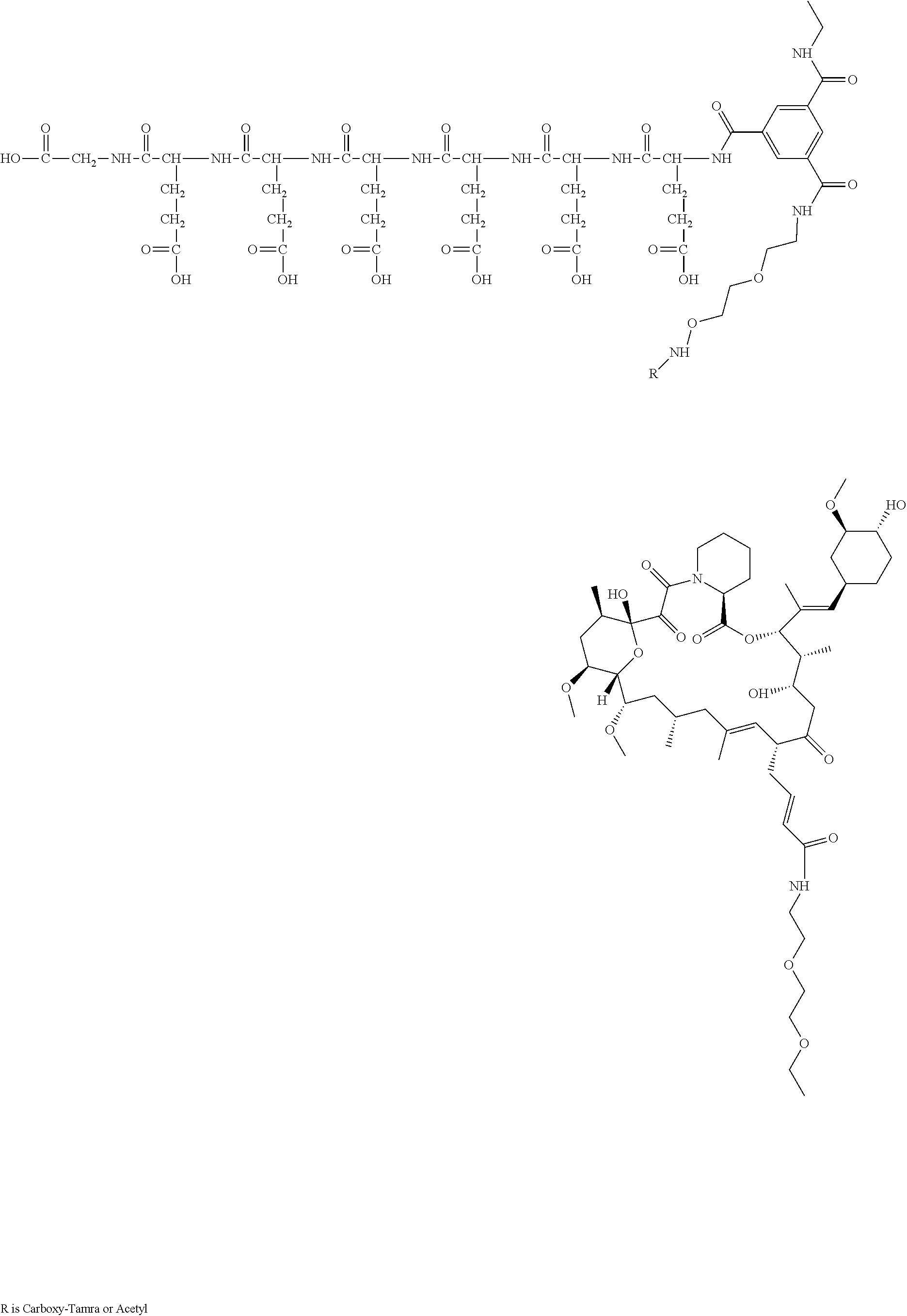
wherein R is a carboxy-tamra or acetyl radical.

27. Active ingredient-peptide construct according to claim 1, characterized in that the construct corresponds to the Formula

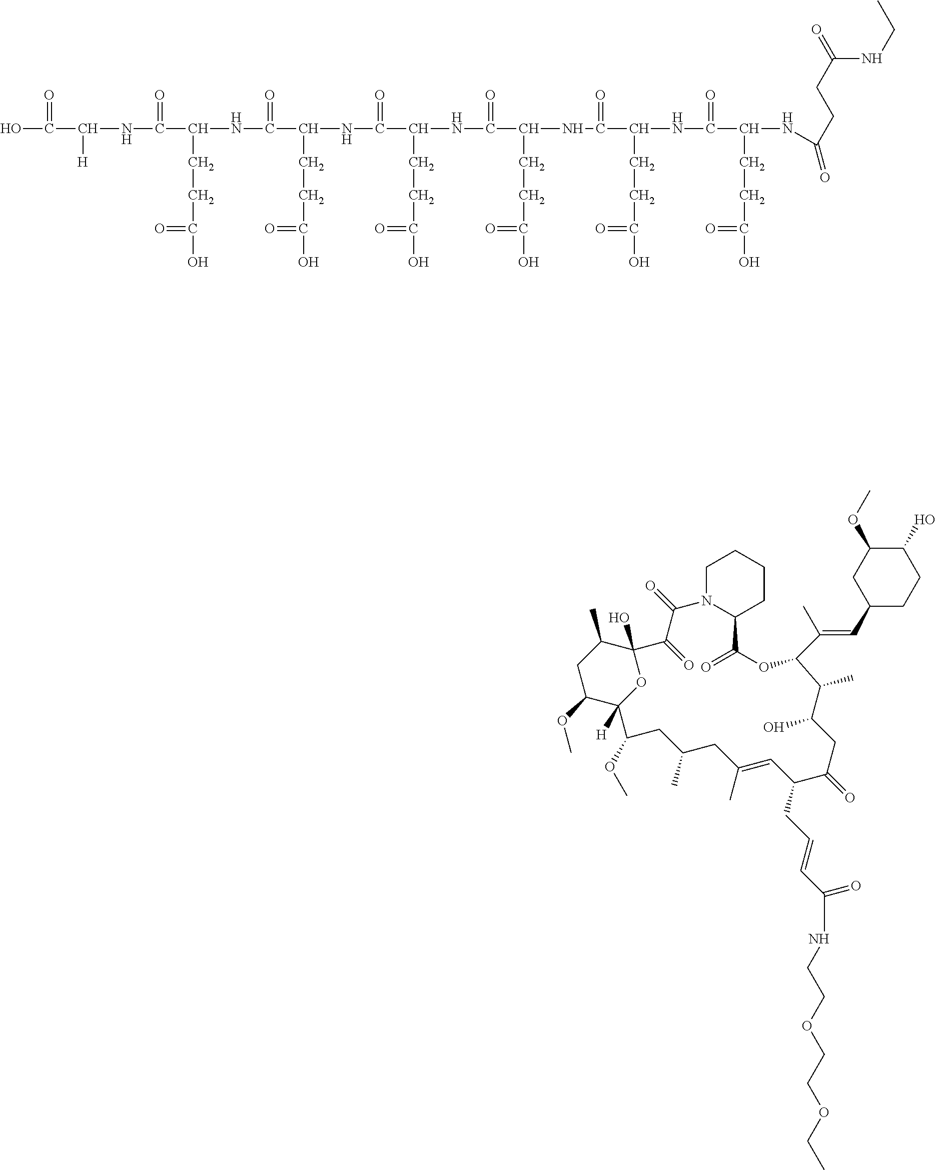

28. Active ingredient-peptide construct according to claim 1, characterized in that the construct corresponds to the Formula

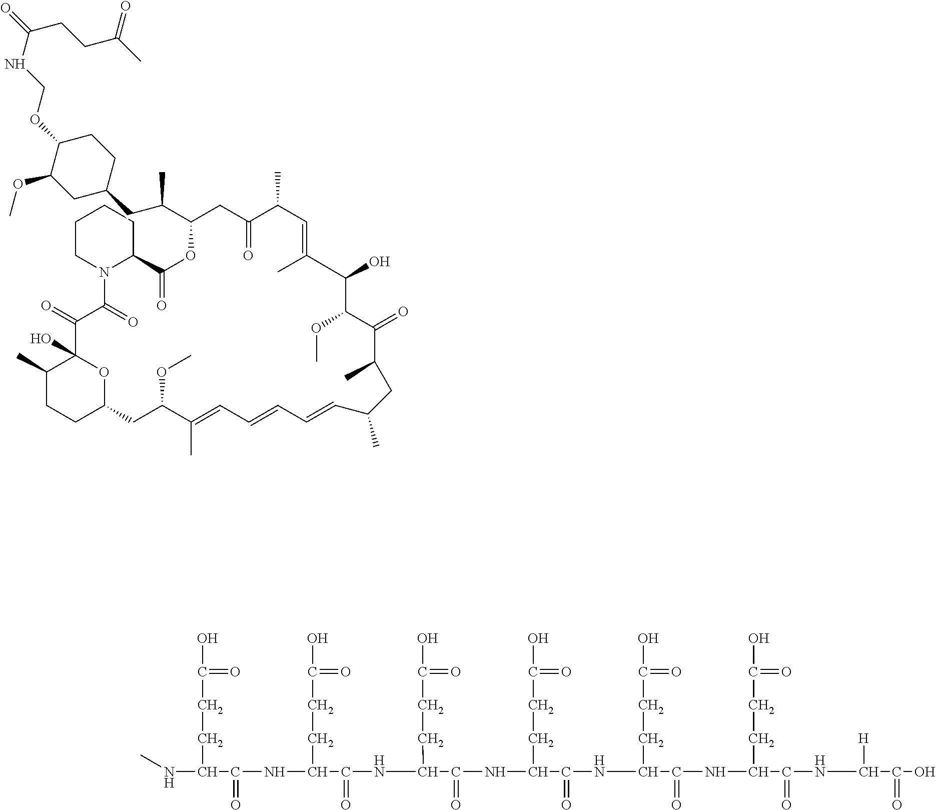

29. Active ingredient-peptide construct according to claim 1, characterized in that the construct corresponds to the Formula 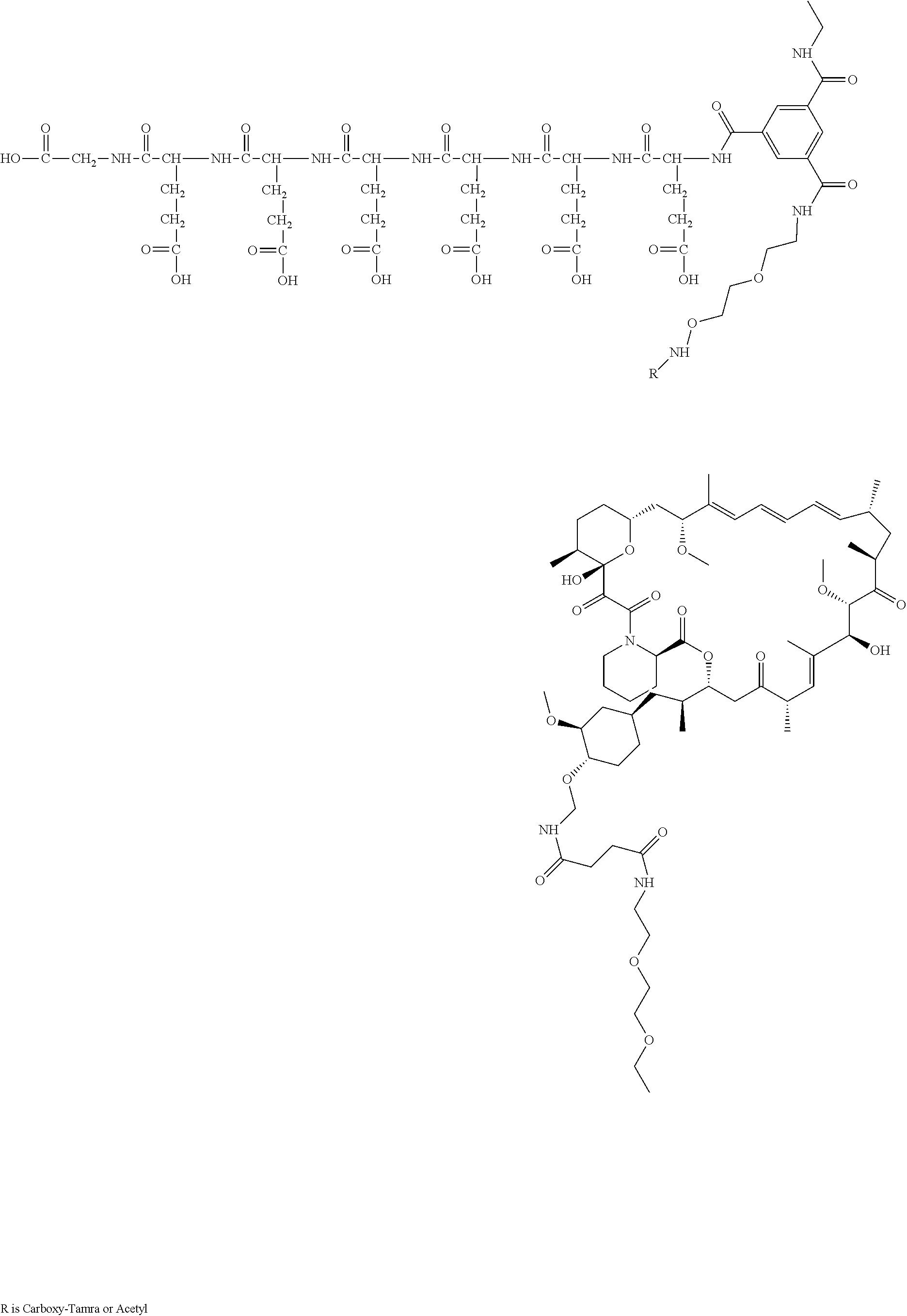
wherein R is a carboxy-tamra or acetyl radical.

30. Process for the concentration of active ingredients in an extracellular space of a multicellular object, comprising the steps:
Providing an active ingredient-peptide construct according to claim 1;
Bringing the construct into contact with a multicellular object, wherein the multicellular object is a separated organ or body part, blood or a blood fraction, a cell culture or a plant.

31. A method which comprises using an active ingredient-peptide construct according to claim 1 as a medicinal product.

32. A method which comprises using an active ingredient-peptide construct according to claim 1 as a diagnostic aid.

33. A method of using an active ingredient-peptide construct according to claim 1 which comprises administering an effective amount of said active ingredient-peptide construct to a patient in need thereof for the treatment of non-immunosuppressive diseases.

34. Pharmaceutical composition, comprising an active ingredient-peptide construct according to claim 1.
